(19) United States
(12) Patent Application Publication
(10) Pub. No.: US 2014/0186294 A1
(43) Pub. Date: Jul. 3, 2014

(54) PHAGOCYTIC ACTIVITY AS A MARKER OF SYNUCLEINOPATHIC DISEASE

(71) Applicants: Shyra Gardai, Hayward, CA (US); Jennifer A. Johnston, Mill Valley, CA (US)

(72) Inventors: Shyra Gardai, Hayward, CA (US); Jennifer A. Johnston, Mill Valley, CA (US)

(73) Assignee: ELAN PHARMACEUTICALS, INC., Cambridge, MA (US)

(21) Appl. No.: 13/988,761

(22) PCT Filed: Nov. 23, 2011
(86) PCT No.: PCT/US11/62135

§ 371 Date: Nov. 27, 2013

Related U.S. Application Data
(60) Provisional application No. 61/417,174, filed on Nov. 24, 2010.
(60) Provisional application No. 61/480,323, filed on Apr. 28, 2011.

(51) Int. Cl. A61K 38/20; A61K 38/17; G01N 33/569

(52) U.S. Cl. CPC A61K 38/2026; G01N 33/56966; A61K 38/1709
USPC 424/85.2; 435/7.1; 514/1.1; 514/44 R

(57) ABSTRACT

The invention provides methods of screening for agents useful in treating or prophylaxis of synucleinopathic disease, methods of diagnosis or prognosis of the same and methods of treatment and prophylaxis. The invention is based in part on the result that cells with phagocytic activity from subjects with synucleinopathic disease have increased levels of alpha synuclein and reduced phagocytic activity and that these processes can be reversed (i.e., synuclein levels decreased and phagocytic levels increased) by treatment with IL-4 among other agents. The increase in phagocytic activity is readily amenable to detection and provides a basis for a screening assay in which an agent is contacted with phagocytic cells and the effect of the agent on phagocytic activity is detected, which is an indicator that the agent has the ability to reduce alpha synuclein levels.

### CROSS-REFERENCE TO RELATED APPLICATION

The present application is a non-provisional claiming the benefit of 61/417,174 filed Nov. 24, 2010 and 61/480,323 filed Apr. 28, 2011, both incorporated by reference in their entirety for all purposes.

### BACKGROUND OF THE INVENTION

Alpha-synuclein brain pathology is a conspicuous feature of several neurodegenerative diseases, including Parkinson's disease (PD), dementia with Lewy bodies (DLB), the Lewy body variant of Alzheimer's disease (LBVAD), multiple systems atrophy (MSA), and neurodegeneration with brain iron accumulation type-1 (NBIA-1). Common to all of these diseases, termed synucleinopathies, are proteinaceous insoluble inclusions in the neurons and the glia which are composed primarily of alpha synuclein.

Lewy bodies and Lewy neurites are intraneuronal inclusions which are composed primarily of alpha-synuclein. Lewy bodies and Lewy neurites are the neuropathological hallmarks Parkinson's disease (PD). PD and other synucleinopathic diseases have been collectively referred to as Lewy body disease (LBD). LBD is characterized by degeneration of the dopaminergic system, motor alterations, cognitive impairment, and formation of Lewy bodies (LBs). (McKeith et al., Clinical and pathological diagnosis of dementia with Lewy bodies (DLB): Report of the CDLB International Workshop, Neurology (1996) 47:1113-24). Other LBDs include diffuse Lewy body disease (DLBD), Lewy body variant of Alzheimer's disease (LBVAD), combined PD and Alzheimer's disease (AD), and multiple systems atrophy. Dementia with Lewy bodies (DLB) is a term coined to reconcile differences in the terminology of LBDs.

Disorders with LBs continue to be a common cause for movement disorders and cognitive deterioration in the aging population (Galasko et al., Clinical-neuropathological correlations in Alzheimer's disease and related dementias. Arch. Neurol. (1994) 51:888-95). Although their incidence continues to increase, creating a serious public health problem, to date these disorders are neither curable nor preventable and understanding the causes and pathogenesis of PD is critical towards developing new treatments (Tanner et al., Epidemiology of Parkinson's disease and akinetic syndromes, Curr. Opin. Neurol. (2000) 13:427-30). The cause for PD is controversial and multiple factors have been proposed to play a role, including various neurotoxins and genetic susceptibility factors.

Specifically, several studies have shown that the synaptic protein alpha-SN plays a central role in PD pathogenesis since: (1) this protein accumulates in LBs (Spillantini et al., Nature (1997) 388:839-40; Takeda et al., AM. J. Pathol. (1998) 152:367-72; Wakabayashi et al., Neurosci. Lett. (1997) 239:45-8), (2) mutations in the alpha-SN gene co-segregate with rare familial forms of parkinsonism (Kruger et al., Nature Gen. (1998) 18:106-8; Polymeropoulos et al., Science (1997) 276:2045-7) and, (3) its overexpression in transgenic mice (Masliah et al., Science (2000) 287:1265-9) and Drosophila (Feany et al., Nature (2000) 404:394-8) mimics several pathological aspects of PD. Thus, the fact that accumulation of alpha-SN in the brain is associated with similar morphological and neurological alterations in species as diverse as humans, mice, and flies suggests that this molecule contributes to the development of PD.

Subjects with Parkinson's have been reported to have elevated pro-inflammatory cytokines, such as IL-6 and IL-1beta, not only in the CSF but also in the brain following autopsy analysis, (Blum-Degen et al., Neurosci. 202, 17-20 (1995), Mogi et al., Neurosci. Lett. 180, 147-150 (1994a), Mogi et al., 165, 208-210 (1994b) and Muller et al., Acta Neurol. Scand. 98, 142-144 (1998). The substantia niagra region of the brain in Parkinson's subjects contains four to five times more microglia compared to other regions of the brain. Although the pathology of Parkinson's is not restricted to this region, it is a region in which pathologic changes significantly impinge on subject symptoms. Several genes associated with the onset of familial Parkinson's which were thought to be primarily neuronal, have recently been found expressed in microglia, (Loeffler et al., Clin. Neuropharm. 17:370-379, Papadopoulos et al. Molec. Cell Neurology 3, 597-612 (2006)). Whether this inflammatory response by microglia is causative or reactionary is subject to debate.

### BRIEF DESCRIPTION OF THE FIGURES

FIG. 1: Microglia isolated from P1-P3 pups containing a Bac construct of human synuclein (line 26) have elevated levels of human synuclein protein compared to littermate controls.

FIG. 2: Microglia isolated from pups containing a human synuclein BAC construct (line 26) or wild type littermates were incubated with 10 micron beads or apoptotic Jurkat T-cells for 90 minutes. Cells were fixed and a phagocytic index calculated by microscopic visualization (n=17+/−s.e.m *p<0.0001)

FIG. 3: Microglia pups or peritoneal macrophages isolated from 12 month old line 26 littermate mice were cultured for 24 hours followed by a 90 minute incubation with apoptotic Jurkat T-cells and a phagocytic index was calculated via microscopic evaluation (n=24 and 18+/−s.e.m *p<0.0001)

FIG. 4: Kidney from 5 line 26 transgenic females were stained for C3, IgG, and IgM and severity of antibody staining was quantified in wild type of synuclein genomic females (n=10+/−sem 0.0063, 0.0077, 0.0039) (H).

FIG. 5: Peritoneal macrophages from 18 month of line 26 mice were cultured in Accell media, Accell media+a pool of targeting human synuclein siRNA, or Accell media+a non targeting siRNA for 3 days. Human synuclein mRNA and protein levels were assessed. Following 3 days of Accell siRNA treatment macrophages were fed 10 uM beads for 90 minutes, fixed, and a phagocytic index calculated (n=14+/−s.e.m *p<0.0001)

FIG. 6: Microglia isolated from individual pups of a wild type by heterozygote cross of line 26, line 422, or line 3 were cultured for 90 minutes with 10 uM beads. A phagocytic index was calculated (+/−s.e.m *p<0.0011, P<0.006, P<0.0095).

FIG. 7: H4 cells were transfected with 25 or 100 ng of wild type, A53T, or A30P mutant forms of synuclein for 2 days followed by the addition of 4 micron bead for 90 minutes. Overexpression of the A53T form of synuclein more potently inhibited phagocytosis at a lower concentration than wild type synuclein.

FIG. 8: Microglia isolated from pups of a wild type by heterozygote cross of line 26, line 422, or line 3 were stimulated with LPS for 18 hr, and TNF-alpha levels were measured by Elisa. Microglia overexpressing synuclein secrete significantly less cytokines upon LPS stimulation than wild type littermate controls.

FIG. 9: Synuclein null or synuclein overexpressing microglia on the murine synuclein null background were stimulated with LPS for 8 hr and cytokine expression was assessed at the mRNA level by multiplex analysis. While synuclein overexpressing release lower levels of cytokines compared to wild type control their response to LPS at the mRNA levels is equivalent to wild type cells.

FIG. 10: Microglia isolated from wild type or synuclein overexpressing pups were fed beads for 90 minutes followed by FM-143 addition on ice for 10 minutes, fluorescence was assessed by flow cytometry. Geometric mean fluorescence of FM-143 from 3 independent experiments was compiled. Wild type microglia demonstrate an increase in plasma membrane volume following phagocytosis, a process which did not occur in microglia isolated from synuclein overexpressing synuclein.

FIG. 11: Rabs proteins were transfected into a stable H4 cells containing a tetracycline inducible synuclein construct. Cells were treated or untreated with tetracycline for 48 hours and then fed 4 μM beads for 90 minutes and a phagocytic index calculated. While overexpression of Rab proteins had little effect of phagocytosis in wild type cells overexpression of Rab3B and Rab11B restored phagocytosis in synuclein overexpressing cells.

FIG. 12: Microarray analysis of microglia isolated from three human donors were stimulated with IL-4 for 24 hours and microarray experiment performed and results were confirmed by qPCR. qPCR was also performed on microglia isolated from IL-4 treated line 26/synuclein null pups. In both instances treatment with IL-4 resulted in a 50% reduction in synuclein mRNA levels.

FIG. 13: Wild type or synuclein overexpressing microglia isolated from line 3, 26, or 422 littermate pups cells were treated for 24 hours with IL-4 and the fed beads for 90 minutes and phagocytic index determined. 11-4 treatment restored phagocytosis in all three genomic lines examined.

FIG. 14: Nucleoporation of microglia with the kinase active G2019S form of LRKK2 induces spontaneous actin rearrangement as measured by increased staining with the F-actin phalloidin dye and induction of microspikes around the cell.

FIG. 15: Overexpression of the G2019S form of LRRK2 enhanced phagocytosis while the wild type form had no effect.

FIG. 16: INF-γ treatment of microglia induced LRRK2 mRNA transcript levels and protein levels by about 2 fold.

FIG. 17: Treatment of microglia for 24 hr with INFγ increased phagocytosis while treatment of cells with INF-γ for 24 followed by a 20 minute treatment with the various LRRK2 inhibitors reduced the phagocytosis of 10 micron bead

FIG. 18: Wild type microglia the G2019S form of LRRK enhanced phagocytosis. In synuclein overexpressing cells it significantly decreased phagocytosis.

FIG. 19: INFγ treatment of wild type microglia enhanced phagocytosis while this same treatment on synuclein overexpressing cells resulted in an even further reduction in phagocytosis.

FIG. 20: Strepavadin tagged fluorescent antibodies detected biotin-coated beads (upper chart); and fluorescent-tagged biotin bound to and identified the strepavadin-coated beads (lower chart).

FIG. 21: Normal microglia were capable of engulfing 5 and 6 micron beads and Cytochalasin D treatment blocked this phagocytosis which was confirmed by biotin-APC binding to the beads.

FIG. 22: A two fold increase in synuclein expression was observed between synuclein overexpressing and wild type cells, similar to what we observe by western blot.

### SUMMARY OF THE CLAIMED INVENTION

The invention provides methods of monitoring synucleinopathic disease or providing an indication of presence, susceptibility to or severity of synucleinopathic disease in a subject. Such methods comprise determining phagocytic activity in a blood sample or other phagocytic cells from a subject. The phagocytic activity is used in monitoring or to provide an indication of presence, susceptibility or severity of synucleinopathic disease in the subject.

The invention further provides methods of providing an indication of presence, susceptibility to or severity of synucleinopathic disease, comprising: determining phagocytic activity of peripheral macrophages of a subject; and comparing the phagocytic activity of peripheral macrophages in the subject to one or more control levels of phagocytic activity, wherein reduced phagocytic activity in the subject relative to phagocytic activity of peripheral macrophages from an undiseased individual is an indicator of presence, susceptibility or extent of synucleinopathic disease. Optionally, the individual has at least one symptom of synucleinopathic disease and the reduced phagocytic activity in combination with the symptom is used to diagnose the subject with synucleinopathic disease. Optionally, the subject lacks symptoms of synucleinopathic disease, and the reduced phagocytic activity is used in assessing susceptibility to synucleinopathic disease. Optionally, the subject has been diagnosed with synucleinopathic disease, and the reduced phagocytic activity is used to assess severity of synucleinopathic disease. Optionally, the methods further comprise contacting the peripheral macrophages of the subject with IL-4 or IL-13 or agonist thereof, and assessing whether the phagocytic activity increases in response to the IL-4 or IL-13 or agonist thereof.

The invention further provides methods of providing an indication of presence, susceptibility or severity of synucleinopathic disease, comprising determining phagocytic activity of peripheral macrophages of a subject in the presence and absence of IL-4 or IL-13 or an agonist thereto, wherein increased activity in the presence of IL-4 or IL-13 or agonist is an indication of the presence, susceptibility or severity of synucleinopathic disease.

The invention further provides methods of monitoring synucleinopathic disease in subject, comprising determining phagocytic activity of peripheral macrophages of the subject at a plurality of times, and associating a change in phagocytic activity, if any, over time with a change in susceptibility or severity of disease. In some methods, the subject has been diagnosed with synucleinopathic disease and increased phagocytic activity over time is associated with reduced severity of disease. In some methods, the subject is being treated with a drug and the plurality of times includes a time before and after initiating administration of the drug, and increased phagocytic activity indicates a positive response to the drug in the subject. In some methods, the subject has not been diagnosed with synucleinopathic disease on commencing monitoring, and decreased phagocytic activity over time is associated with increased susceptibility to disease. In any such method, alpha synuclein expression in the phagocytic cells can also be determined; wherein the phagocytic activity and/or the level of alpha synuclein provide an indication of susceptibility or severity of the synucleinopathic disease.

The invention further provides methods of monitoring synucleinopathic disease in subject, comprising determining phagocytic activity of blood samples from the subject at a plurality of times, and associating a changes in phagocytic activity, if any, with changed susceptibility to or severity of the disease. In some methods, the subject has been diagnosed with synucleinopathic disease and increased phagocytic activity over time is associated with reduced severity of disease. In some methods, the subject is being treated with a drug and the plurality of times includes a time before and after initiating administration of the drug, and increased phagocytic activity indicates a positive response to the drug in the subject. In some methods, the subject has not been diagnosed with synucleinopathic disease on commencing monitoring, and decreased phagocytic activity over time is associated with increased susceptibility to disease. In some methods, alpha synuclein expression in cells from the blood sample is also determined; wherein the phagocytic activity and/or the level of alpha synuclein provide an indication of susceptibility or severity of the synucleinopathic disease.

The invention further provides methods of providing an indication of presence, susceptibility to or severity of synucleinopathic disease, comprising: determining phagocytic activity in a blood sample from a subject; and comparing the phagocytic activity of the blood sample from the subject to one or more control levels of phagocytic activity, wherein reduced phagocytic activity in the subject relative to phagocytic activity of a blood sample from an undiseased individual is an indicator of presence, susceptibility or extent of synucleinopathic disease. Optionally, the individual has at least one sign or symptom of synucleinopathic disease and the reduced phagocytic activity in combination with the symptom is used to diagnose the subject with synucleinopathic disease. Optionally, the subject lacks symptoms of synucleinopathic disease, and the reduced phagocytic activity is used in assessing susceptibility to synucleinopathic disease. Optionally, the subject has been diagnosed with synucleinopathic disease, and the reduced phagocytic activity is used to assess severity of synucleinopathic disease. Optionally, the methods further comprise contacting the peripheral macrophages of the subject with IL-4 or IL-13 or agonist thereof, and assessing whether the phagocytic activity increases in response to the IL-4 or IL-13 or agonist thereof.

The invention further provides methods of providing an indication of presence, susceptibility or severity of synucleinopathic disease, comprising: determining phagocytic activity of a blood sample from a subject in the presence and absence of IL-4 or IL-13 or an agonist thereto, wherein increased activity in the presence of IL-4 or IL-13 or agonist is an indication of the presence, susceptibility or severity of synucleinopathic disease.

The invention further provides methods of monitoring synucleinopathic disease in subject, comprising determining phagocytic activity of blood samples from the subject at a plurality of times, and associating increased phagocytic activity with a reduction of severity of the disease. Optionally, the subject is being treated with a drug and the plurality of times includes a time before and after initiating administration of the drug, and increased phagocytic activity indicates a positive response to the drug in the subject.

The invention further provides the use of phagocytic activity in diagnosis, prognosis or monitoring of synucleinopathic disease, optionally, wherein the phagocytic activity is measured in a blood sample.

The invention further provides methods of treating or effecting prophylaxis of synucleinopathic disease, comprising administering to a subject having or at risk of synucleinopathic disease IL-4 or a nucleic acid encoding IL-4 or an agonist of IL-4. Optionally, the agonist is a zinc finger protein or nucleic acid encoding the same, wherein the zinc finger protein binds to and stimulates transcription of IL-4.

The invention further provides methods of treating or effecting prophylaxis of synucleinopathic disease, comprising administering rab3b or rab11b or a nucleic acid encoding either of these, or an agonist of either of these. Optionally, the agonist is a zinc finger protein that binds to and stimulates transcription of rab3b or rab11b.

The invention provides methods of providing an indication of presence, susceptibility to or severity of synucleinopathic disease. Such methods comprise determining phagocytic activity of phagocytic cells of a subject, wherein alpha synuclein expression in the phagocytic cells is also determined; wherein the phagocytic activity and/or the level of alpha synuclein provide an indication of presence, susceptibility to or severity of synucleinopathic disease. Optionally, the methods further comprises determining the level of alpha synuclein in the phagocytic cells. Optionally, the cells include peripheral macrophages or polymorphonuclear cells. Optionally, the phagocytic activity is determined from a first fluorescent signal and the expression of alpha synuclein from a second fluorescent signal. Optionally, the first and second fluorescent signals are detected simultaneously. Optionally, the first and second fluorescent signals are detected by FACS. Optionally, the phagocytic activity is determined from uptake of fluorescently labeled cells or beads and the alpha synuclein expression is determined from uptake of a fluorescently labeled antibody that specifically binds to intracellular alpha synuclein. Optionally, the methods further comprise comparing the phagocytic activity of the phagocytic cells in the subject to one or more control levels of phagocytic activity of phagocytic cells from an undiseased individual. Optionally, the methods further comprise comparing the alpha synuclein expression of the phagocytic cells in the subject to one or more control levels of alpha synuclein expression of phagocytic cells from an undiseased individual. Optionally, the method further comprises comparing the phagocytic activity and the alpha synuclein expression of the phagocytic cells in the subject to one or more control levels of phagocytic activity and alpha synuclein expression of phagocytic cells from an undiseased individual. The comparing can be performed for example in a computer programmed to perform the comparing and provide output of an indication of presence, susceptibility to or severity of synucleinopathic disease. Optionally, the synucleinopathic disease is sporadic Parkinson's disease. The subject can be a G2019S carrier or a non-carrier. In some methods, reduced phagocytic activity and/or increased alpha synuclein expression in the subject relative to phagocytic activity and alpha synuclein expression of phagocytic cells from an undiseased individual is an indicator of presence, susceptibility or extent of synucleinopathic disease. In some methods, the individual has at least one sign or symptom of synucleinopathic disease and the reduced phagocytic activity and/or increased alpha synuclein expression in combination with the symptom is used to diagnose the subject with synucleinopathic disease. In some methods, the subject lacks symptoms of synucleinopathic disease, and the reduced phagocytic activity and/or increased alpha synuclein expression is used in assessing susceptibility to synucleinopathic disease. In some methods, the subject has been diagnosed with synucleinopathic disease, and the reduced phagocytic activity and/or increased alpha synuclein expression is used to assess severity of synucleinopathic disease. In some methods, reduced phagocytic activity provides an indication that the subject is affected with synucleinopathic disease. In some methods, increased phagocytic activity provides an indication that the subject is unaffected with synucleinopathic disease.

The invention further provides methods of providing an indication of presence, susceptibility to or severity of synucleinopathic disease, comprising determining phagocytic activity of cells in a blood sample of a subject, wherein alpha synuclein expression in cells of a blood sample from the subject is also determined; wherein the phagocytic activity and/or the level of alpha synuclein provides an indication of presence, susceptibility to or severity of synucleinopathic disease. Some methods further comprise determining the level of alpha synuclein in cells of the blood sample. In some methods, the phagocytic activity and the alpha synuclein expression are determined on the same blood sample. In some methods, the phagocytic activity and the alpha synuclein expression are determined on the same or overlapping population of cells in the blood sample. In some methods, the cells include peripheral macrophages. In some methods, the cells include polymorphonuclear cells. In some methods, the phagocytic activity is determined from a first fluorescent signal and the expression of alpha synuclein from a second fluorescent signal. In some methods, the first and second fluorescent signals are detected simultaneously. In some methods, the first and second fluorescent signals are detected by flow cytometry. In some methods, the phagocytic activity is determined from uptake of fluorescently labeled cells or beads and the alpha synuclein expression is determined from uptake of a fluorescently labeled antibody that specifically binds to intracellular alpha synuclein. Some methods further comprise comparing the phagocytic activity of the blood sample in the subject to one or more control levels of phagocytic activity of a blood sample from an undiseased individual. Some methods further comprise comparing the alpha synuclein expression of the blood sample in the subject to one or more control levels of alpha synuclein expression of a blood sample from an undiseased individual. Some methods further comprise comparing the phagocytic activity and the synuclein expression of the blood sample in the subject to one or more control levels of phagocytic activity and alpha synuclein expression of a blood sample from an undiseased individual. In some methods, the comparing is performed in a computer programmed to perform the comparing and provide output of an indication of presence, susceptibility to or severity of synucleinopathic disease. In some methods, the synucleinopathic disease is sporadic Parkinson's disease. The subject can be a G2019S carrier or non-carrier. In some methods, reduced phagocytic activity and/or increased alpha synuclein expression in the subject relative to phagocytic activity and alpha synuclein expression of a blood sample from an undiseased individual is an indicator of presence, susceptibility or extent of synucleinopathic disease. In some methods, the individual has at least one sign or symptom of synucleinopathic disease and the reduced phagocytic activity and/or increased alpha synuclein expression in combination with the symptom is used to diagnose the subject with synucleinopathic disease. In some methods, the subject lacks symptoms of synucleinopathic disease, and the reduced phagocytic activity and/or increased synuclein expression is used in assessing susceptibility to synucleinopathic disease. In some methods, the subject has been diagnosed with synucleinopathic disease, and the reduced phagocytic activity and/or increased alpha synuclein expression is used to assess severity of synucleinopathic disease. In some methods, reduced phagocytic activity provides an indication that the subject has symptomatic synucleinopathic disease. In some methods, increased phagocytic activity provides an indication the subject does not have symptomatic synucleinopathic disease.

The invention further provides methods of differentially treating subjects with a synucleinopathic disease, comprising: determining phagocytic activity in phagocytic cells or a blood sample of the subjects; and treating subjects with below normal phagocytic activity with a regime and treating subjects with normal or above normal phagocytic activity with a different regime.

The invention further provides methods for selecting candidate human subjects for participation in a clinical trial involving a drug for treating a synucleinopathic disease, comprising determining the phagocytic activity of phagocytic cells or a blood sample of the human subjects, and segregating the subjects for inclusion or exclusion in the trial based on the level of phagocytic activity. In some methods, individuals with below normal phagocytic activity are included in the trial, and individuals with normal or above normal level are excluded. In some methods, the candidate human subjects have a 2019 mutation of LRRK2.

The invention further provides methods of screening an LRRK2 binder or modulator comprising: contacting a test agent with a phagocytic cell over-expressing alpha synuclein having reduced phagocytic activity relative to a control cell without alpha synuclein overexpression; and determining whether the test agent increases the phagocytic activity of the cell, an increase providing an indication that the agent is useful in inhibiting LRRK2. In some methods, the phagocytic cells have a 2019 mutation in LRRK2. Some methods further comprise contacting the cell with IFN-γ. Some methods further comprise contacting the enantiomer of the test agent with the phagocytic cell; determining whether the enantiomer increases the phagocytic activity of the cell, an increase in the phagocytic activity by the test agent and unchanged phagocytic activity by the enantiomer provide an indication that the test agent is useful in inhibiting LRRK2.

The invention further provides methods of screening an LRRK2 binder or modulator, comprising: contacting a test agent with a phagocytic cell having an LRRK2 2019 mutation and/or treated with IFN-γ, wherein the phagocytic cell does not overexpress alpha synuclein and has increased phagocytic activity relative to a control cell without an LRRK2 2019 mutation and not treated with IFN-γ; and determining whether the test agent decreases the phagocytic activity of the cell, a decrease providing an indication that the agent is useful in inhibiting LRRK2. Some methods further comprise contacting the enantiomer of the test agent with the phagocytic cell treated with IFN-γ; and determining whether the enantiomer decreases the phagocytic activity of the cell, a decrease in the phagocytic activity by the test agent and unchanged phagocytic activity by the enantiomer providing an indication that the test agent is useful in inhibiting LRRK2. In some methods, the determining comprises contacting the phagocytic cells with inert particles or apoptotic cells and counting uptake of the particles or cells in the phagocytic cell. In some methods, the cell is a microglial cell or a peripheral macrophage.

The invention provides methods of screening a test agent for activity useful in treatment of synucleinopathic disease comprising, contacting a test agent with a phagocytic cell containing an exogenous gene expressing alpha synuclein or from a subject with synucleinopathic disease; and determining whether the test agent increases the phagocytic activity of the cell, an increase providing an indication that the agent is useful in treatment of synucleinopathic disease. Optionally, the determining comprises contacting the phagocytic cells with inert particles or apoptotic cells and counting uptake of the particles or cells in the phagocytic cell. Optionally, the cell is a microglial cell or a peripheral macrophage from a subject with synucleinopathic disease or a transgenic animal with an alpha synuclein transgene. Optionally, the cell is a neuronal cell transfected with alpha synuclein. Optionally, the methods further comprise determining the alpha synuclein content of the cell before and after contacting the cell with the test agent. Optionally, the methods further comprise administering the test agent to an animal model of synucleinopathic disease and determining whether the test agent inhibits, reduces or delays at least one sign or symptom of synucleinopathic disease.

The invention further provides a diagnostic kit comprising an entity that can be phagocytosed and an antibody to alpha synuclein. The entity to be phagocytosed can be a labeled inert particle or apoptotic cell.

### DEFINITIONS

Specific binding refers to the binding of a an agent to a target (e.g., a component of a sample) that is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however imply that an agent binds one and only one target. Thus, an agent can and often does show specific binding of different strengths to several different targets and only nonspecific binding to other targets. Specific binding usually involves an association constant of 10⁷, 10⁸ or 10⁹ M⁻¹ or higher.

The term “antibody” or “immunoglobulin” is used to include intact antibodies and binding fragments thereof. Typically, fragments compete with the intact antibody from which they were derived for specific binding to an antigen. Fragments include separate heavy chains, light chains, Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody. A bispecific or bifunctional antibody is an artificial hybrid antibody having two different heavy/light chain pairs and two different binding sites. The term “antibody” also includes single-chain antibodies in which heavy and light chain variable domains are linked through a spacer.

The term “subject” includes human and other mammalian subjects. The term can refer to an individual anywhere on a spectrum from having no signs or symptoms of disease and to an individual with full symptoms of disease. Individuals in this spectrum can progress from being asymptomatic to having one or more suns of disease to one or more symptoms to full-blown disease. Signs and symptoms of disease can develop sequentially or concurrently. Individuals at any of these stages may or may not have genetic or other known risk of developing the disease.

A subject is at known risk of developing a disease if the subject does not yet have the disease as conventionally defined (e.g., by Diagnostic and Statistical Manual IV TR) but has a known risk factor (e.g., genetic mutation, family history, occupational) predisposing subjects with that risk factor to a significantly higher chance of developing the disease than subjects (optionally aged-matched subjects) without the risk factor.

Susceptibility refers to probability or risk of developing a disease and/or imminence of developing the disease. Susceptibility can be a relative term comparing a subject individual with a control individual or population (e.g., undiseased individuals) or comparing a series of measurements on the same patient. A higher susceptibility means a higher risk and/or a shorter period between measurement and onset of disease. For example, in a presently asymptomatic individual, a reduced level of phagocytosis and/or increased level of alpha synuclein relative to undiseased control individuals indicates a higher risk of developing disease than the control individuals. To further illustrate, in a series of measurements on an asymptomatic individual, decreasing phagocytosis levels and/or increasing phagocytosis levels indicate both increased risk and shorter time from the last measurement to onset of symptomatic disease.

The term “symptom” refers to a subjective evidence of a disease, such as altered gait, as perceived by the subject. A “sign” refers to objective evidence of a disease as observed by a physician (e.g., reduced level of phagocytosis or increased level of alpha synuclein either in the form of Lewy bodies, in a body fluid, or intracellularly within phagocytic cells).

An isolated agent or other moiety means that the moiety if found in nature is separated at least in part from the molecules with which it is naturally associated including flanking sequences if the peptide is part of a longer protein. If the peptide or moiety is synthetic, isolated means separated at least in part from chemicals used in its production. An isolated agent does not exclude the presence of heterologous components, such as pharmaceutical excipients not naturally associated with the agent or used in its synthesis. An isolated agent can also be pure (e.g., at least 50, 75, 90 or 99% w/w pure) of contaminants. An isolated macromolecular agent can also be the predominant macromolecular species in a composition.

The term “agent” includes any compound including compounds with or without pharmaceutical activity, natural compounds, synthetic compounds, small molecules, peptides and peptidomimetics.

The term “pharmacologic agent” means an agent having a pharmacological activity. Pharmacological agents include compounds that are known drugs, compounds for which pharmacological activity has been identified but which are undergoing further therapeutic evaluation in animal models or clinical trials. An agent can be described as having pharmacological activity if it exhibits an activity in a screening system that indicates that the active agent is or may be useful in the prophylaxis or treatment of a disease. The screening system can be in vitro, cellular, animal or human. Agents can be described as having pharmacological activity notwithstanding that further testing may be required to establish actual prophylactic or therapeutic utility in treatment of a disease.

An exogenous gene is a gene not normally found in a cell or animal (e.g., a human gene in a transgenic mouse), or a gene occurring at a different genomic location than normal. The term gene includes genomic sequences, cDNA sequence, mini-genes and the like.

Statistical significance implies a p value 0.05.

A G2019S carrier means a subject who is homozygous or heterozygous for a G2019S mutation of LRRK2. Such carriers account for about 1% of Parkinson's disease patients. Both heterozygous and homozygous mutations are associated with a known risk of Parkinson's disease. Because the G2019S mutation is associated with a broad range of disease onset (about 30-75) and incomplete penetrance, carriers can be symptomatic or asymptomatic.

The terms “peripheral macrophage” and “monocyte’ have the same meaning in this application.

### DETAILED DESCRIPTION OF THE INVENTION

### I. General

The invention provides methods of screening for agents useful in treating or prophylaxis of synucleinopathic disease, methods of diagnosis or prognosis of the same and methods of treatment and prophylaxis. The invention is based in part on the result that cells with phagocytic activity from subjects with synucleinopathic disease have increased levels of alpha synuclein and reduced phagocytic activity and that these processes can be reversed (i.e., alpha synuclein levels decreased and phagocytic levels increased) by treatment with IL-4 among other agents. The increase in phagocytic activity is readily amenable to detection and provides a basis for a screening assay in which an agent is contacted with phagocytic cells and the effect of the agent on phagocytic activity is detected, which is an indicator that the agent has the ability to reduce alpha synuclein levels. An ability to reduce intracellular levels of alpha synuclein is useful for treatment and prophylaxis of synucleinopathic disease, which is characterized by such intracellular deposits of alpha synuclein. Reduced phagocytic activity, optionally in combination with increased alpha synuclein level, is also a useful diagnostic or prognostic indicator in a subject. The reduced phagocytic activity, alone or in combination with an increased alpha synuclein level, provides an indication of presence of, risk of developing and/or severity of synucleinopathic disease in a subject. The reduced phagocytic activity can be detected in peripheral macrophages of a subject. These cells are present in the peripheral blood in contrast to other potential markers of synucleinopathic disease present in the brain or CNS, which are much less accessible. Alpha synuclein levels can also be measured in cells from peripheral blood, the same or different from the phagocytic cells used to assess phagocytosis.

Although practice of the invention is not dependent on an understanding of mechanism, it is believed that increased intracellular levels of alpha synuclein interfere with vesicle trafficking and the impaired vesicle trafficking gives rise to or at least contributes to synucleinopathic disease. Impaired vesicle trafficking also contributes to reduced phagocytic activity. Because vesicle trafficking is related to both disease pathology and phagocytic activity, impairment of phagocytic activity serves as an indicator of disease pathology. Impairment of phagocytic activity may also have a direct effect on disease pathology through reduced capacity to clear products of neuronal degeneration.

### II. Phagocytic Cells

The screening and diagnostic assays employ cells with phagocytic activity, sometimes referred to as phagocytic cells. The cells can be human, or other mammalian, particular rodent or mouse. The cells can be obtained from a subject with synucleinopathic disease (usually a human) or a transgenic animal model of disease (e.g., a mouse). The cells can also be a cell line transformed to express an alpha synuclein gene. The cells include microglial cells or peripheral macrophages or polymorphonuclear cells or other phagocytic cells. Microglia can be obtained by biopsy or differentiating or trans-differentiation of embryonic stems cells, bone marrow, induced pluripotent or other stem or pluripotent cell sources from a subject or transgenic animal model. Such phagocytic cells have higher intracellular levels of alpha synuclein that control cells from an undiseased individual. Some neuronal cells with phagocytic activity can also be obtained from such a subject, by surgical biopsy or after death or differentiating or trans-differentiation of embryonic stems cells, bone marrow, induced pluripotent or other stem or pluripotent cell sources. For diagnostic assays, phagocytic cells assayed are preferably from a blood sample as discussed further below, but other sources of phagocytic cells from a subject can also be used. All of these cell types (i.e., microglia, peripheral macrophages and neuronal cells) can also be obtained from transgenic animal models of synucleinopathic disease (i.e., expressing an alpha synuclein transgene). These cells obtained from such a source have higher intracellular levels of alpha synuclein than control cells of the same type from an otherwise comparable control nontransgenic animal. Phagocytic cells can also be obtained by transforming primary cells or cell lines obtained from undiseased individuals or nontransgenic animals so that they express alpha synuclein at higher levels than comparable nontransformed cells. One example of a suitable cell line is the H4 human neuronal cell line (ATCC HTB-148™; Amstein, J. Natl. Cancer Inst. 52: 71-84, 1974; Day, Nature 279: 797-799 1979) transformed with a construct encoding alpha synuclein.

Some phagocytic cells include a G2019S or other mutated form of LRRK2. Such cells can be obtained by transformation of a cell line or transgenic animal with LRRK2 including a G2019S mutation, optionally together with a second transgene encoding alpha synuclein. Such cell lines can also be obtained from humans with a G2019S mutation, who may be carriers as yet without symptoms of Lewy body disease or may have symptoms. Depending on whether the cells are co-transfected with alpha synuclein or obtained from a G2019S carrier with symptomatic Parkinson's disease, such cells may or may not have elevated levels of alpha synuclein. As discussed in more detail below, concurrent presence of a G2019S mutation and elevated levels of alpha synuclein, as occurs in a symptomatic G2019S carrier, is associated with reduced phagocytosis whereas a G2019S mutation without elevated levels of alpha synuclein (e.g., from cells not transfected with alpha synuclein or an asymptomatic G2019S carrier, is associated with increased levels of phagocytosis relative to the mean level in non-diseased individuals.

### III. Screening Assay

The components of a screening assay include cells with phagocytic activity, a test agent being screened and an entity that can be phagocytosed. Such an entity can be inert particles, such as latex beads, preferably of size 4-10 microns. The entity can also be apoptotic cells, such as Jurkat cells). Cells and inert entities are phagocytosed by different mechanisms, the former being receptor dependent. Usually the cells are contacted with the test agent first and incubated for a period (e.g., 1-48 hr) before adding the entity to be phagocytosis. Phagocytosis can be followed by light microscopy or flow cytometry among other methods detecting uptake of the entity being phagocytosed into the phagocytic cell. Methods for detection of phagocytosis can include any fluorescent based platform (e.g., flow cytometry, microscopy, array scan, or a Tr-fret, fluorescent plate reader) enzymatic, or colorimetric readout.

An increase in phagocytic activity can be assessed relative to a base line value before contacting phagocytic cells with a test agent or with a control reaction in which the test agent is absent. Such controls are negative controls. Increased phagocytic activity can also be assessed relative to a positive control, such as an agent known to stimulate phagocytic activity of cells with decreased levels of alpha synuclein (e.g., IL-4). A similar or greater stimulation of phagocytic activity relative to IL-4 indicates provides an indication that a test agent has useful activity in stimulating phagocytic cells. In some of the examples below, an enantiomer of a test agent is used as a negative control. A differential effect between the test agent and its enantiomer (e.g., the test agent increases phagocytosis and the enantiomer does not) indicates the test agent acts via a target specific effect, such as inhibition of LRRK2. An additional control reaction can be performed to detect and subtract any background level of beads bound to the surface of cells but not internalized as described in the Examples.

The screening assay can be performed on different types of phagocytic cells in parallel or sequentially. Some agents stimulate phagocytic activity in all or multiple cell types. Other agents may stimulate phagocytic activity in some cells type but not all. For example, IL-4 is effective in stimulating phagocytic activity in microglial cells and peripheral macrophages but not in H4 neuronal cells, the lack of stimulation in the H4 cells being due to lack of an IL-4 receptor.

The increased phagocytic activity detected by the above assay serves as a surrogate marker for decreased intracellular concentration of alpha synuclein and consequently rescued vesicle trafficking, which is a desired pharmacological activity for treatment of synucleinopathic disease. The increase in phagocytosis may alternatively or additionally be due to alteration in alpha synuclein localization, or ability to interact with members of the vesicle machinery Reduced intracellular levels of alpha synuclein can also be assessed directly at the mRNA and/or protein levels in such assays on the same, overlapping or distinct population of cells as that on which phagocytosis is assayed. To analyze mRNA, the phagocytic cells are lysed and mRNA analyzed by probe hybridization (e.g., to a probe array) or quantitative PCR among others. Alternatively levels of alpha synuclein protein can be assessed by a intracellular staining following by flow cytometry, or immunological assay, such as a Western blot or ELISA (or Luminex analysis). Reduced levels of mRNA encoding alpha synuclein or alpha synuclein protein relative to baseline measurements before contacting phagocytic cells with a test agent indicate a desired pharmacological activity, as do comparable or greater levels relative to a positive control, such as IL-4.

Agents to be screened can include agents known or suspected of being agonists or mimics of IL-4 (e.g., agonistic antibodies to the IL-4 receptor), IL-13, or peptide mimics or IL-4 or IL-13. One agent reported to mimic IL-4 is the transcription factor STAT6, Kamogawa et al., J. Immunol. 161(3):1074-7 (1998). Two helix coiled coil peptide mimetics of IL-4 incorporating a leucine-zipper domain of the yeast transcription factor GCN4 as a scaffold into which the putative binding epitope of IL-4 for IL-4R alpha was transferred in a stepwise manner are described by Domingues et al., Nat. Struct. Biol. 6(7):652-6 (1999). A further IL-4 agonist termed DHP-14-AB based pm a four-helix designed protein is described by Laporte et al., Proc. Natl. Acad. Sci. USA. 102(6):1889-94 (2005). Natural products to be screened can also be obtained from the National Cancer Institute's Natural Product Repository, Bethesda, Md. Random libraries of peptides or other agents can also be screened for suitability. Combinatorial libraries can be produced for many types of agents that can be synthesized in a step-by-step fashion. Such agents include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated herein by reference for all purposes). Peptide libraries can also be generated by phage display methods. See, e.g., Devlin, WO 91/18980.

One class of test agents that can be screened, particularly in cells with a G2019S mutations, is known binders or modulators of LRRK2, several of which are known. Such modulators can agonize or antagonize LRRK2, preferably the latter. Such compounds include antibodies to LRRK2, siRNA inhibiting expression of LRRK2, dominant negative variants of LRRK2 among others. Other examples of LRRK2 inhibitors include GW5074 (3-(3,5-Dibromo-4-hydroxy-benzylidene)-5-iodo-1,3-dihydro-indol-2-one; Lee et al., Nature Medicine 16:998-1000, 2010), and those reported in US2010/0273769. LRRK2 inhibitors may preferentially inhibit wild type LRRK2 or preferentially inhibit to G2019S LRRK2 or inhibit both. Test agents can also include agents known or suspected of inhibiting other kinases, such as cFMS or PLK2.

Screening assays can also be performed on phagocytic cells with enhanced LRRK2 activity, as a result of G2019 mutation or treatment with an agonist, such as interferon gamma. If such cells also overexpress alpha synuclein (e.g., from transformation or as a result of being obtained from a symptomatic patient with Lewy body disease), then phagocytic activity is reduced relative to control cells without enhanced LRRK2 activity, and compounds can be screened as described as above. Such methods are particularly useful as a secondary screen for agents that are known to bind LRRK2 and/or inhibit its kinase activity in vitro. As with the assays described above, increased phagocytic activity resulting from treatment with an agent relative to a control provides an indication the agent has useful activity for treatment of synucleinopathic disease via inhibition of LRRK2. Optionally, the control can be an enantiomer of the agent being tested. In this case, stimulation of phagocytosis by the agent but not the enantiomer or increased stimulation by the agent relative to the enantiomer provides an indication that the agent acts by direct inhibition of LRRK2 rather than by a secondary effect on a pathway involving LRRK2.

Screening assays can also be performed on phagocytic cells with enhanced LRRK2 activity that do not overexpress alpha synuclein. Such cells can be obtained from cell lines or transgenic animals transformed with LRRK2 G2019S but not alpha synuclein or from individuals who are carriers of G2019S but not symptomatic or from phagocytic cells treated with an agonist of LRRK2 such as gamma interferon. Such cells show enhanced phagocytosis relative to otherwise comparable control cells having normal LRRK2 activity (i.e., a wildtype gene and not treated with an agonist). Such cells can be contacted with agents known or suspected to be LRRK2 binders or inhibitors. In this case, LRRK2 inhibition can be seen by a reduction in phagocytosis resulting from treatment with the agent relative to a control.

### IV. Animal Models

A number of transgenic animal models of synucleinopathic disease have been described in the scientific and patent literature. In general, such animals have a transgene encoding alpha synuclein in operable linkage with a promoter expressed in neuronal cells. Such animal models are disposed to develop at least one sign or symptom of synucleinopathic disease, such as alpha synuclein aggregates or Lewy body like structures formed from alpha synuclein. Examples of animal models are described in U.S. Pat. No. 6,504,080, Gispert et al., Mol. Cell. Neurosci. 24, 419-429 (2003), Feany et al, Nature 404, 393-398 (2000), WO 00/20020, WO 01/60794, WO 03/015507, U.S. Pat. No. 6,504,080). A transgenic animal incorporating a genomic alpha synuclein transgene used in the present examples is described in more detail in co-pending commonly assigned Ser. No. 11/352,403. Transgenic animal models are useful in screening agents identified by in vitro assays to confirm activity in inhibiting, delaying or reducing at least one sign or symptom of synucleinopathic disease. Transgenic animal models can also be used as a source of phagocytic cells for phagocytic assays as described above.

### V. Diagnostic, Prognostic and Monitoring Assays

The invention further provides an assay for diagnosing, prognosing, monitoring or assessing the severity of individuals having or at risk of having synucleinopathic disease as further defined herein. The assay can be performed on an individual anywhere on a spectrum from having no signs or symptoms of disease and to an individual with symptoms (and signs) of full-blown disease. Individuals in this spectrum can progress from being asymptomatic to having one or more signs of disease to one or more symptoms but not yet full-blown disease to full-blown disease (e.g., meeting DSM IV TR criteria). Signs and symptoms of disease can develop sequentially or concurrently. Individuals at any of these stages may or may not have genetic or other known enhanced risk of developing the disease.

The assay can be performed on a fluid sample removed and typically not returned to the subject. The sample can be a blood sample or CNS sample. The assay is performed on phagocytic cells from such an individual. Preferably the cells are obtained from the peripheral blood (e.g., peripheral macrophages or polymorphonuclear cells or other phagocytic cells) because such cells are available with minimally invasive techniques (i.e., simple drawing of blood). Such as assay can be performed on whole blood or any fraction thereof containing cells with phagocytic activity, preferably containing peripheral macrophages. The sample, whether blood or CNS, may or may not be subject or processing before performing the assay, but cells containing phagocytic activity should remain for performing the assay.

Cells drawn from such a subject (e.g., in the form of a blood or CNS sample) are contacted with entities that can be phagocytosed (e.g., latex bead or apoptotic cells) and phagocytic activity is measured relative to that of otherwise comparable cells from an individual or population of individuals not having or at known risk of synucleinopathic disease (i.e., undiseased individuals). Significantly decreased phagocytic activity under of a test subject relative to the norm in undiseased individuals (e.g. less than 1 or 2 standard deviations below the mean) provides an indication of presence, susceptibility (e.g., imminence or probability of development of symptoms of), or degree of synucleinopathic disease. Conversely, normal levels (e.g., within 1 or 2 standard deviations of the mean) or above normal levels provides an indication of absence of symptomatic synucleinopathic disease. Such an indication can be used together with other signs or symptoms of synucleinopathic disease in diagnosing synucleinopathic disease. Such an indication, optionally in combination with other sign(s) and/or symptom(s) of synucleinopathic disease, can provide an indication of susceptibility including risk and/or imminence of developing synucleinopathic disease in a currently asymptomatic individual or in individuals having sign(s) or symptom(s) consistent with the disease but not yet by themselves sufficient for diagnosis. The methods for example can be used on a subject with a known risk factor, such as a genetic mutation, and some sign(s) and/or symptom(s) that are consistent with but the disease but also consistent with other diagnoses. The methods can also be used on individuals lacking a known risk factor but with some such sign(s) and/or symptom(s). In an individual with diagnosed synucleinopathic disease, the level of phagocytic activity can provide an indication of severity of disease, a lower level indicating more severe disease. The methods can also be used on an individual lacking any known risk factor and lacking any signs or symptoms of synucleinopathic disease. The levels can also be used to monitor treatment with an increased level of phagocytosis relative to a baseline before beginning treatment or no or less (relative to deterioration in untreated patients) reduction in level after commencing treatment providing an indicating treatment is achieving a desired result.

Phagocytosis level can also be used in monitoring subjects for progression to onset of disease. Before commencing such monitoring, a subject may have no known signs or symptoms of synucleinopathic disease or may have one or more signs or symptoms but insufficient for a diagnosis of synucleinopathic disease to be made. A reduced level of phagocytosis over time indicates increased susceptibility to synucleinopathic disease. Conversely, maintaining a constant level indicates the same or reduced susceptibility to synucleinopathic disease, as does an increased level of phagocytic activity.

Such diagnostic, prognostic or monitoring assays can be performed in the presence and absence of an agent known to stimulate phagocytic activity of phagocytic cells with increased levels of alpha synuclein, such as IL-4 or IL-13. Rescue of a reduced level of phagocytic activity in cells from a subject being tested with IL-4, IL-13 or similar known stimulator provides a further indication of reduced phagocytic activity in the individual and in consequence, increased levels of intracellular alpha synuclein and thus presence, susceptibility, or severity of synucleinopathic disease in a subject.

Assessment of phagocytosis in diagnosis, prognosis or monitoring can be combined with assessment of alpha synuclein levels. The alpha synuclein level measured is preferably a measure of intracellular alpha synuclein but can also be a measure of soluble alpha synuclein in a body fluid, such as blood or plasma. The assessment of alpha synuclein levels can be performed on the same or different sample as the assessment of phagocytosis. An intracellular alpha synuclein level and phagocytosis level can be assessed on the same cell population, overlapping populations or distinct populations. Preferably, phagocytosis and alpha synuclein levels are both assessed from cells in a peripheral blood cell. As in other analyses, cells use for the respective analyses in the blood sample can be the same, overlapping or different populations. The analyses of phagocytosis and synuclein levels can be performed on either order or concurrently. In some methods, phagocytosis and alpha synuclein levels are measured concurrently on the same cells. Concurrent analysis can be performed by for example differential labeling of cells or beads taken up by phagocytosis and of alpha-synuclein and differential detection, e.g., by microscopy, e.g., an ARRAYSCAN™ fluorescent scan imager, flow cytometry or FACS®.

Analysis of a phagocytosis is sometimes performed with a control to distinguish entities (e.g., beads, cells or other particles) taken up into cells from those bound to the surface. After uptake of entities, a labeled control molecule is contacted with the cells under conditions such that the control molecule binds to any entities on the surface but is not taken up itself significantly. Such can be achieved by assessing phagocytosis of fluorescent entities labeled with biotin or strepavidin and a control molecule that is differentially labeled streptavidin or biotin respectively. Entities taken up by cells and beads on the surface of cells can then be distinguished by differential labeling. If an intracellular level of alpha synuclein is also detected, it can also be differentially labeled, such that three different labels are present, one for intracellular beads, one for surface-bound beads and one for intracellular alpha synuclein. The labels can be detected simultaneously or sequentially or in any combination or order.

Elevated levels of intracellular synuclein, and particularly in combination with reduced levels of phagocytosis provide an indication of presence, susceptibility, or severity of onset of synucleinopathic disease. For example, a higher level of intracellular alpha synuclein indicates a higher susceptibility or severity. A level of intracellular alpha synuclein is elevated if increased beyond at least one or two standard deviations of the mean in undiseased individuals. In general, a combination of decreased phagocytosis and increased intracellular synuclein, particularly when measured in the same blood sample, is more strongly associated with presence, susceptibility, or severity to onset of synucleinopathic disease than either indicator alone.

Similar principles apply in performing diagnostic assays in G1920S carriers as other patients except that in asymptomatic carriers phagocytosis levels may be significantly elevated (e.g., greater than one or two standard deviations over the mean in undiseased individuals) rather than at normal levels. In this case, reduced phagocytosis and/or increased alpha synuclein levels are still an indicator of presence, susceptibility or imminence of symptomatic disease. However, detection of an elevated phagocytosis and/or normal alpha synuclein is an indicator that the carrier is asymptomatic and not in imminent danger of developing symptomatic disease.

Monitoring the level of phagocytosis in such a patient, optionally in combination with measuring alpha synuclein level, can be used as a measure of progression or lack thereof from asymptomatic to symptomatic status. For example, a patient who starts with elevated phagocytosis and progresses through normal phagocytosis to reduced phagocytosis is indicated as progressing from asymptomatic to symptomatic status. A patient who has stable elevated levels of phagocytosis is indicated as remaining asymptomatic. A patient who starts with elevated levels of phagocytosis and proceeds to normal levels is indicated as being imminently close to developing symptomatic disease. Although it might be thought that the patient status could be assessed from development of symptoms alone, in facts, early symptoms of Parkinson's disease can be hard to distinguish from those of other diseases or hypochondria in those knowing they are at risk from the disease due to a genetic mutation (or otherwise), and the availability of an objective indicator alone or in combination with assessment of symptoms if any provide a more accurate diagnosis or prognosis of the patient.

The above methods of diagnosis, prognosis or monitoring are useful notwithstanding that as with other such methods the diagnosis, prognosis or monitoring is not always completely accurate. Such methods are useful provided they increase the probability of accurate diagnosis, prognosis, or monitoring compared with the situation in which the assays were not performed. Accuracy can sometimes be increased by performing the above methods in combination with other methods, (e.g., conventional monitoring of signs and symptoms of disease). In general, diagnosis indicates a present state of the patient (e.g., presence of synucleinopathic disease), prognosis is a prediction of development of a future state (e.g. synucleinopathic disease, and monitoring a series of measurements (i.e., at least 2) to assess a change in state over time (e.g., response to treatment). However, as used in this application as in the art, the terms overlap in meaning. For example, detection of decreased phagocytosis and/or elevated alpha synuclein in a patient not having full-blown signs and symptoms of Parkinson's disease can be viewed as diagnostic of a presently abnormal but inchoate stage of disease as well as prognostic of future development of the full-blown disease. Likewise, by indicating a positive response to treatment, monitoring can be predictive of a future state of the patient (e.g., reduced severity or less rapid deterioration). Thus, the same measurement can sometime be used for any or all of assessing present state, future state or changes in a patient.

As indicated above phagocytic activity and alpha synuclein level can be measured simultaneously or separately. Phagocytic activity can be measured from uptake of labeled beads followed by for example, microscopy or flow cytometry. The alpha synuclein level can be detected at the protein level by for example, microscopy, ELISA, Western blot, flow cytometry or FACS, or at the rRNA level by probe hybridization or quantitative PCR. Preferably, alpha synuclein is detected by staining of intracellular alpha synuclein in cells and detection of the cells by flow cytometry or FACS. If the staining is with a macromolecule, such as an antibody to alpha synuclein, cells are preferably permeabilized, e.g., as described in the Examples.

Components of the diagnostic assays (e.g., bead being phagocytosed or antibody binding to intracellular alpha synuclein can be detectably labeled. A detectable label refers to an atom (e.g., radionuclide), molecule (e.g., fluorescein), enzyme, or complex, that is or can be used to detect (e.g., due to a physical or chemical property), or indicate the presence of a target to which the detectable label is bound. Binding can be direct as when a label is bound to a bead or indirect as when intracellular alpha synuclein is labeled via binding of an antibody that is itself labeled. Beads with various fluorescent labels incorporated into the bead material itself are commercially available. Useful detectable labels include biotin for staining with labeled streptavidin conjugate or vice versa, fluorescent dyes (e.g., fluorescein, Texas red, rhodamine, green fluorescent protein, enhanced green fluorescent protein, and the like), radiolabels (e.g., ³H, ¹²⁵I, ³⁵S, ¹⁴C, or ³²P), enzymes (e.g., hydrolases, particularly phosphatases such as alkaline phosphatase, esterases and glycosidases, or oxidoreductases, particularly peroxidases such as horse radish peroxidase, and others commonly used in ELISAs), substrates, cofactors, inhibitors, chemiluminescent groups, chromogenic agents, and colorimetric labels such as colloidal gold or colored glass or plastic, (see, e.g., U.S. Pat. Nos. 3,817,837; 3,850,752; 3,939,350; 3,996,345; 4,277,437; 4,275,149; and 4,366,241) Radiolabels and chemiluminescent labels can be detected using photographic film or scintillation counters, fluorescent markers may be detected using a photodetector to detect emitted light (e.g., as in flow cytometry or fluorescence-activated cell sorting). Enzymatic labels are typically detected by providing the enzyme with a substrate and detecting the reaction product produced by the action of the enzyme on the substrate, and colorimetric labels are detected by simply visualizing the colored label. Thus, a label is any composition detectable by spectroscopic, photochemical, biochemical, immunochemical, electrical, optical or chemical means

Assays can be automated, such that signals indicative of phagocytosis level and/or alpha synuclein are received by a digital computer programmed to receive and analyze signals representative of the levels of an analyte in an assay, such as by comparing them with a mean signal in undiseased individuals, and provide output of results (i.e., phagocytosis levels and/or alpha synuclein levels). The result may be presented for example, as a chart illustrating the level of phagocytosis in a patient compared with a range (e.g., +/−one or two standard deviations around the mean) of that in undiseased individuals. The output may or may not also indicate a diagnosis, prognosis or monitoring conclusion (e.g., indication of symptomatic disease; positive response to treatment, progressing from asymptomatic to symptomatic disease). A suitable computer can include a central processing unit for performing calculations, a display for displaying output, interface, a keyboard, a pointing device, main memory storing various programs, and a storage device that can store data received from an assay, programming or output data. The computer can be a personal computer, a digitally enabled television, cell phone, personal digital assistant, or the like. Information residing in the main memory or storage device can be used to program such a system and can represent, for example, a disk-type optical or magnetic media, magnetic tape, solid state dynamic or static memory.

### VI. Proteins

The application refers to several proteins include alpha synuclein, IL-4, IL-13, rab3b, rab11b. Unless otherwise apparent from the context, the human form of such proteins is meant. Species variants (e.g., mammalian) or induced variants (e.g., at least 90% sequence identity) of human alpha synuclein can also be used but a human sequence is preferred. Exemplary sequences from the Swiss Prot Database are alpha synuclein (P37840), IL-4 (P05112), IL-13 (P35225), rab3b (P20337) and rab11b Q15907. The Swiss Prot database also lists several allelic and species variants of the human sequences. Some known natural variants of human alpha synuclein include E46K, A30P and A53T (the first letter indicates the amino acid in the wild type Swiss-Prot sequence, the number is the codon position in the Swiss Prot sequence, and the second letter is the amino acid in the allelic variant). Forms of alpha synuclein including combinations of these natural variants can also be made.

LRRK2 or leucine-rich repeat kinase 2 refers to a protein including an ankyrin repeat region, a leucine-rich repeat (LRR) domain, a kinase domain, a DFG-like motif, a RAS domain, a GTPase domain, an MLK-like domain, and a WD40 domain or to the gene encoding the same. The protein is present largely in the cytoplasm but also associates with the mitochondrial outer membrane. Accession numbers for mammalian LRRK2 sequences in the NCBI database include: AAV63975.1 (human), XP_{—}001168494.1 (Pan troglodytes), XP_{—}615760.3 (Bos Taurus), XP_{—}543734.2 (Canis familiaris), NP 080006.2 (Mus musculus), and XP_{—}235581.4 (Rattus norvegicus). A number of naturally occurring mutations of human LRRK2 are associated with Parkinson's Disease. A 6055A mutation in exon 41 of the LRRK2 gene leads to a G2019S amino acid substitution of a highly conserved residue within the predicted activation loop of the MAPKKK (Mitogen-Activated Protein Kinase) domain. This mutation enhances the protein kinase activity of LRRK2 (see, e.g., WO2008/122789). Other LRRK2 mutations include R1441C, R1441G, Y1699C R1914H, 12012T, 12020T, or G2385R. R1441C, R1441G, Y1699C or T2356I have similar protein kinase activity to wild-type LRRK2. R1914H, 12012T and G2385R are nearly inactive. 12020T has intermediate activity between wild-type LRRK2 and R1914H or 12012T. A human form of LRRK2, optionally with a natural mutation or combination of mutations, preferably a G1920S mutation is preferably used in the present methods.

### VII. Subjects Amenable to Treatment, Diagnosis, Prognosis and Monitoring Regimes

Subjects amenable to treatment, diagnosis, prognosis or monitoring include individuals at risk of a synucleinopathic disease but not showing signs and/or symptoms, as well as subjects presently showing one or more symptoms. Synucleinopathic disease means a disease characterized by excess levels of alpha synuclein or abnormal pathological characteristics including alpha synuclein relative to normal (undiseased) individuals. Such diseases include all forms of Parkinson's disease (including forms of disease with known genetic abnormalities and idiopathic Parkinson's disease), DLB, DLBD, LBVAD, pure autonomic failure, Lewy body dysphagia, incidental LBD, inherited LBD (e.g., mutations of the alpha-SN gene, PARK3 and PARK4) and multiple system atrophy (e.g., olivopontocerebellar atrophy, striatonigral degeneration and Shy-Drager syndrome). The present treatment methods can be administered prophylactically to individuals who have a known risk of a synucleinopathic disease (e.g., genetic or biochemical) but who are asymptomatic or at least have symptoms insufficient for diagnosis of Parkinson's disease. Such individuals include those having relatives who have experienced this disease, and those whose risk is determined by analysis of genetic or biochemical markers. Genetic markers of risk toward PD include mutations in the alpha synuclein or Parkin, UCHLI, CYP2D6 genes and LRRK2 genes; particularly mutations at position 53 of the alpha synuclein gene and a G2019S mutation in LRRK2. Individuals presently suffering from Parkinson's disease can be recognized from its clinical manifestations including resting tremor, muscular rigidity, bradykinesia and postural instability. Other secondary and nonmotor symptoms that affect many people and are increasingly recognized by doctors as important to recognizing Parkinson's (e.g., stooped posture, dystonia, fatigue, impaired fine motor dexterity and motor coordination, impaired gross motor coordination, poverty of movement, akathisia, speech problems, loss of facial expression, micrographia, difficulty swallowing, sexual dysfunction, cramping, and drooling). Some subjects experience tremor as their primary symptom, whereas others may not have tremors, but may have problems with balance. Also, for some subjects the disease progresses quickly, and in others it does not. These and other risk factors or signs and symptoms of Parkinson's degrees can also provide a reason to perform a diagnostic or prognostic assay of the invention as described above. The diagnostic or prognostic assays also provide a means to identify patients at known risk of developing or having a synucleinopathic disease for prophylactic or therapeutic treatment.

In asymptomatic subjects, treatment can begin at any age (e.g., 10, 20, or 30). Usually, however, it is not necessary to begin treatment until a subject reaches 40, 50, 60, or 70. Optionally, presence of absence of symptoms, signs or risk factors of a disease is determined before beginning treatment.

### VIII. Therapeutic Regimes

Agents that can be used in therapeutic regimes include IL-4, IL-13, nucleic acids encoding the same or agonists thereto, rab3b, rab11b, nucleic acids encoding the same or agonists thereto. One class of agonist are zinc finger proteins binding to an activating any of IL-4, IL-13, rab3b or rab11b. Another class of antagonists are anti-idiotypic antibodies to IL-4 or IL-13. Agents also include agents with activity in stimulating phagocytosis or decreasing intracellular levels of alpha synuclein identified by the screening methods described above.

In prophylactic applications, pharmaceutical compositions or medicaments are administered to a subject at known risk of or otherwise susceptible to developing a synucleinopathic disease, in a regime (i.e., dose, frequency, route of delivery) sufficient to at last reduce the risk, lessen the severity, or delay the onset of the disease, including biochemical, histological and/or behavioral symptoms of the disease, its complications and intermediate pathological phenotypes presenting during development of the disease.

In therapeutic applications, compositions or medicaments are administered to a subject suspected of, or already suffering from such a disease in a regime (dose, frequency, route) sufficient to reduce, or at least slow deterioration of the symptoms of the disease (biochemical, histological, and/or behavioral), including its complications and intermediate pathological symptoms. An amount adequate to accomplish therapeutic or prophylactic treatment is defined as a therapeutically- or prophylactically-effective dose. In therapeutic regimes, the agent is usually administered at intervals until symptoms of the disease disappear or significantly decrease. Optionally administration can be continued to prevent recurrence. In prophylactic regimes, agents are also usually administered at intervals, in some instances for the rest of a subject's life. Treatment can be monitored by assaying levels of administered agent, or by monitoring the response of the subject.

Effective doses of the compositions of the present invention, for the treatment of the above-described conditions vary depending upon many different factors, including means of administration, target site, physiological state of the subject, whether the subject is human or an animal, other medications administered, and whether treatment is prophylactic or therapeutic. Usually, the subject is a human; nonhuman mammals, including transgenic mammals, can also be treated. Treatment dosages are typically titrated to optimize safety and efficacy.

Dosages of antibodies, peptides, and small molecules range from about 0.0001 to about 100 mg/kg, and more usually about 0.01 to about 20 mg/kg, of the host body weight. An exemplary treatment regime entails administration once per day, week, every two weeks or once a month or once every 3 to 6 months. The dosage and frequency of administration can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until the progression of the disease is reduced or terminated, and preferably until the subject shows partial or complete amelioration of the symptoms of the disease. In some instances, the subject can be administered the same regime as for prophylactic administration.

Doses for nucleic acid encoding agents range from about 10 ng to 1 g, about 100 ng to about 100 mg, about 1 ng to about 10 mg, or about 30 to about 300 μg DNA per subject. Doses for infectious viral vectors may vary from about 10 to about 100, or about 10³, about 10⁴, about 10⁵, about 10⁶, about 10⁷, about 10⁸, about 10⁹, about 10¹⁰, or more virions per dose.

Agents of the invention can be administered by parenteral, topical, intravenous, oral, subcutaneous, intrathecal, intraarterial, intracranial, intraperitoneal, intranasal, or intramuscular means for prophylactic and/or therapeutic treatment. In some methods, agents are injected directly into a particular tissue where deposits have accumulated, for example, intracranial injection.

Agents of the invention can optionally be administered in combination with other agents that are at least partly effective in the treatment of synucleinopathic disease. Such agents include antibodies to alpha synuclein or fragments of alpha synuclein that induce such antibodies. Sinemet (Levodopa/Carbidopa), dopamine agonists such as Requip and Mirapex, Symmetrel, Artane, Cogentin, Eldepryl (also known as Deprenyl), Tasmar, and Comtan.

Agents of the invention are often administered as compositions comprising an active therapeutic agent and a variety of other pharmaceutically acceptable components. See Remington's Pharmaceutical Science (15th ed., Mack Publishing Company, Easton, Pa., 1980). The particular formulation employed depends on the intended mode of administration and the therapeutic application. The compositions can also include, depending on the formulation desired, pharmaceutically acceptable, non toxic carriers or diluents, which are defined as vehicles commonly used to formulate pharmaceutical compositions for animal or human administration. The diluent is selected so as not to negatively impact the biological activity of the combination. Examples of such diluents include, but are not limited to, distilled water, physiological phosphate-buffered saline, Ringer's solution, dextrose solution, and Hank's solution. In addition, the pharmaceutical composition or formulation may also include other carriers, adjuvants, or nontoxic, nontherapeutic, nonimmunogenic stabilizers, and the like.

Pharmaceutical compositions can also include large, slowly metabolized macromolecules such as proteins, polysaccharides such as chitosan, polylactic acids, polyglycolic acids, copolymers (such as latex functionalized SEPHAROSE® beads, agarose, cellulose, and the like), polymeric amino acids, amino acid copolymers, and lipid aggregates (such as oil droplets or liposomes).

For parenteral administration, agents of the invention can be administered as injectable dosages of a solution or suspension of the substance in a physiologically-acceptable diluent with a pharmaceutical carrier that can be a sterile liquid such as water, oils, saline, glycerol, or ethanol. Parenteral compositions for human administration are sterile, substantially isotonic, and made under GMP conditions. Additionally, auxiliary substances, such as wetting or emulsifying agents, surfactants, pH buffering substances, and the like, can be present in compositions. Other components of pharmaceutical compositions are those of petroleum, animal, vegetable, or synthetic origin, for example, peanut oil, soybean oil, and mineral oil. In general, glycols, such as propylene glycol or polyethylene glycol, are preferred liquid carriers, particularly for injectable solutions.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or microparticles such as polylactide, polyglycolide, or copolymer for enhanced adjuvant effect, as discussed above (see Langer, Science, 249:1527 33 (1990) and Hanes et al., Advanced Drug Delivery Reviews, 28:97-119 (1997). The agents of this invention can be administered in the form of a depot injection or implant preparation that can be formulated in such a manner as to permit a sustained or pulsatile release of the active ingredient.

Additional formulations suitable for other modes of administration include oral, intranasal, and pulmonary formulations, suppositories, and transdermal applications. Intranasal delivery is particularly useful for delivering peptides to the brain. Peptides can be formulated, for example, in sterile water, as a nasal spray. For suppositories, binders and carriers include, for example, polyalkylene glycols or triglycerides; such suppositories can be formed from mixtures containing the active ingredient in the range of about 0.5% to about 10%, or about 1% to about 2%. Oral formulations can include excipients such as pharmaceutical grades of mannitol, lactose, starch, magnesium stearate, sodium saccharine, cellulose, and magnesium carbonate. These compositions typically take the form of solutions, suspensions, tablets, pills, capsules, sustained release formulations or powders and contain about 10% to about 95% of active ingredient, or about 25% to about 70%.

Topical application can result in transdermal or intradermal delivery. Topical administration can be facilitated by co administration of the agent with cholera toxin or detoxified derivatives or subunits thereof or other similar bacterial toxins (See Glenn et al., Nature, 391:851 (1998)). Co-administration can be achieved by using the components as a mixture or as linked molecules obtained by chemical cross linking or expression as a fusion protein. Alternatively, transdermal delivery can be achieved using a skin patch or using transferosomes (Paul et al., Eur. J. Immunol., 25:3521 24 (1995); Cevc et al., Biochem. Biophys. Acta, 1368:201 15 (1998)).

Zinc finger proteins can be engineered or selected to bind to any desired target site within a desired gene and activate or repress transcription of that gene depending on the type of regulatory domain attached to the zinc finger protein. For example, a zinc finger protein can be designed to bind to and activate transcription of 11-4, IL-13, rab3b or rab11b. An exemplary motif characterizing one class of zinc finger proteins (C2H2 class) is -Cys-(X)2-4-Cys-(X)12-His-(X)3-5-His (where X is any amino acid). A single finger domain is about 30 amino acids in length, and contains an alpha helix containing the two invariant histidine residues and two invariant cysteine residues in a beta turn coordinated through zinc. The target site can be within a promoter or enhancer or within a structural gene. A zinc finger protein can be linked to a transcriptional repressor, such as the KRAB repression domain from the human KOX-1 protein to suppress transcription (Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994)). Alternatively, a zinc finger protein can be linked to a transcriptional activator, such as VIP 16 to activate transcription. Methods for selecting target sites suitable for targeting by zinc finger proteins, and methods for designing zinc finger proteins to bind to selected target sites are described in WO 00/00388. Methods for selecting zinc finger proteins to bind to a target using phage display are described by EP.95908614.1. The target site used for design of a zinc finger protein is typically of the order of 9-19 nucleotides. Zinc fingers can be administered as a protein but are more commonly administered as a nucleic acid by a gene therapy approach and expressed in situ in subject.

A number of viral vector systems for delivering of nucleic acids encoding therapeutic agents are available including retroviral systems (see, e.g., Lawrie and Tumin, Cur. Opin. Genet. Develop. 3, 102-109 (1993)); adenoviral vectors (see, e.g., Bett et al., J. Virol. 67, 5911 (1993)); adeno-associated virus vectors (see, e.g., Zhou et al., J. Exp. Med. 179, 1867 (1994)), viral vectors from the pox family including vaccinia virus and the avian pox viruses, viral vectors from the alpha virus genus such as those derived from Sindbis and Semliki Forest Viruses (see, e.g., Dubensky et al., J. Virol. 70, 508-519 (1996)), Venezuelan equine encephalitis virus (see U.S. Pat. No. 5,643,576) and rhabdoviruses, such as vesicular stomatitis virus (see WO 96/34625) and papillomaviruses (Ohe et al., Human Gene Therapy 6, 325-333 (1995); Woo et al., WO 94/12629 and Xiao & Brandsma, Nucleic Acids. Res. 24, 2630-2622 (1996)).

DNA encoding an immunogen, or a vector containing the same, can be packaged into liposomes. Suitable lipids and related analogs are described by U.S. Pat. No. 5,208,036, U.S. Pat. No. 5,264,618, U.S. Pat. No. 5,279,833, and U.S. Pat. No. 5,283,185. Vectors and DNA encoding an immunogen can also be adsorbed to or associated with particulate carriers, examples of which include polymethyl methacrylate polymers and polylactides and poly(lactide-co-glycolides), (see, e.g., McGee et al., J. Micro Encap. 1996).

Gene therapy vectors or naked DNA can be delivered in vivo by administration to an individual subject, typically by systemic administration (e.g., intravenous, intraperitoneal, nasal, gastric, intradermal, intramuscular, subdermal, or intracranial infusion) or topical application (see e.g., U.S. Pat. No. 5,399,346). Such vectors can further include facilitating agents such as bupivacine (see e.g., U.S. Pat. No. 5,593,970). DNA can also be administered using a gene gun. See Xiao & Brandsma, supra. The DNA encoding an immunogen is precipitated onto the surface of microscopic metal beads. The microprojectiles are accelerated with a shock wave or expanding helium gas, and penetrate tissues to a depth of several cell layers. For example, The ACCEL Gene Delivery Device manufactured by Agacetus Inc. Middleton, Wis. is suitable. Alternatively, naked DNA can pass through skin into the blood stream simply by spotting the DNA onto skin with chemical or mechanical irritation (see WO 95/05853).

In a further variation, vectors encoding immunogens can be delivered to cells ex vivo, such as cells explanted from an individual subject (e.g., lymphocytes, bone marrow aspirates, and tissue biopsy) or universal donor hematopoietic stem cells, followed by reimplantation of the cells into a subject, usually after selection for cells which have incorporated the vector.

### IX. Clinical Treatments and Differential Treatment Regimes

Assessment of phagocytic activity provides a basis for stratifying individuals for inclusion in a clinical trial. For example, candidates for the clinical trials can be screened for phagocytic activity, and subjects having below normal phagocytic activity selected for inclusion in the trial. Patients can be further stratified by also determining levels of alpha synuclein (as described above) and selecting patients for the trial having both below normal phagocytic activity and above normal alpha synuclein. Patients can additionally or alternatively be stratified by selection based on being a carrier for a genetic mutation associated with synucleinopathic disease, such as G1920S in LRRK2. Selection of such patients also having reduced levels of phagocytosis (and optionally increased alpha synuclein) is useful for therapeutic testing of a drug. Selection of such patients having above normal levels of phagocytosis is useful for testing prophylactic treatment with a drug. Selection of such patients having normal levels of phagocytosis is useful for testing prophylactic or early disease stage treatment.

Similar principles apply in determining whether to treat or which treatment regime to apply to a patient. Patients having reduced levels of phagocytosis are indicated as having symptomatic disease and may therefore be indicated to receive any available treatment for symptomatic Parkinson's disease including those described in the application and otherwise known. Patients having normal levels of phagocytosis or in the case of G1920S normal or above normal levels of phagocytosis can be administered no pharmacological therapy or pharmacological therapy known to effective and appropriate (i.e., not having substantial side effects) in prophylaxis or early stage disease.

### X. Kits

The invention further provides kits including any of the reagents described herein for performing the methods of diagnosis, prognosis, monitoring or screening described. One such kit combines one or more reagents for detecting phagocytosis with one or more reagents for detecting alpha synuclein expression. The one or more reagents for detecting phagocytosis can include, for example, an entity that can be phagocytosed, such as an inert particle, or apoptotic cell. Such an entity is preferably labeled. The one or more reagents for detecting alpha synuclein expression can include an antibody to alpha synuclein or other moiety with this binding specificity. The reagent is also preferably labeled or a secondary label such as a labeled anti-idiotypic antibody is included. Such kits can be used in diagnosis, prognosis or monitoring as described above. Another kit includes an entity that can be phagocytosed and a phagocytic cell. The kit may also include a positive control compound that stimulates phagocytosis of the entity by the cell. Such kits can be used in screening agents for therapeutic activity as disclosed above. Kits can also include packaging or other labeling providing instructs for performing methods, such as screening, diagnosis, prognosis or monitoring to be performed with a kit.

### EXAMPLES

### Example 1

### Overexpression of Endogenous Synuclein in Microglia and Macrophages Disrupts Phagocytosis

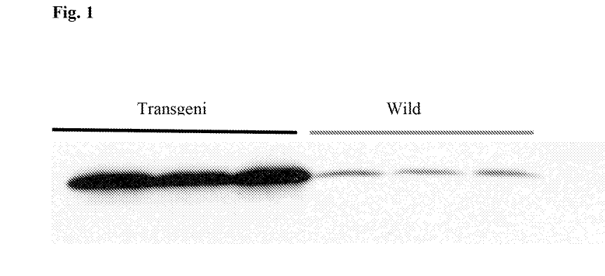

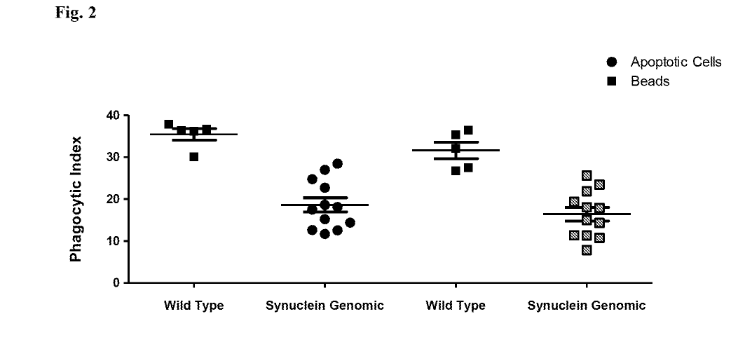

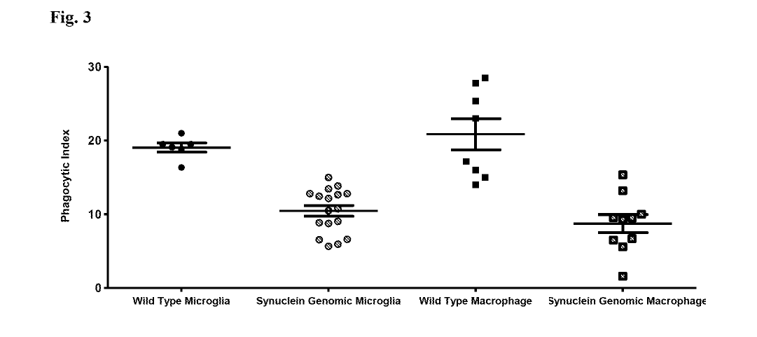

To investigate the role of endogenous synuclein on microglia function, we utilized a transgenic mouse model in which wild type or the E46K mutated form of human synuclein was overexpressed from a human bacterial artificial chromosome (bac). The bac construct contained the synuclein promoter and upstream sequences (45 kbp), as well as downstream 3′ sequences (15 bp). Three animal lines of interest were identified; line 422 expresses wild type synuclein and two additional lines, line 26 and line 3, express the mutant E46K form. Altered microglia function associated with over expression of human synuclein was assessed in cells isolated from line 26 P1-P3 pups due in part to the high synuclein expression observed in this line. FIG. 1. shows that microglia isolated from line 26 genomic pups expressed 4-5 times more synuclein than microglia from littermate wild type pups, a signal absent in microglia isolated from synuclein null animals. Significant overexpression of human synuclein in the murine microglia allowed us to assess the consequence of elevated synuclein levels on microglia function. Phagocytosis of apoptotic cells requires engagement of specific receptors whereas ingestion of inert particles such as latex beads does not. Although the requirement for receptor engagement differs, both targets require activation of intracellular signaling, actin rearrangement, and mobilization of membrane to the phagocytic cup. To evaluate changes in general phagocytic processes microglia were fed 10 μM beads or apoptotic Jurkat T-cells. Overexpression of human synuclein significantly impaired microglia phagocytosis of both beads and apoptotic cells compared with non-transgenic littermate controls as shown in FIG. 2. As both forms of phagocytosis were impaired it suggested not a defect in receptor expression or function, but rather alterations in general phagocytic processes. Reduction in phagocytic function associated with synuclein overexpression was not restricted to microglia or to cells isolated during early development as peritoneal macrophages isolated from adult line 26 animals exhibited defects in phagocytosis to the same magnitude as shown in FIG. 3.

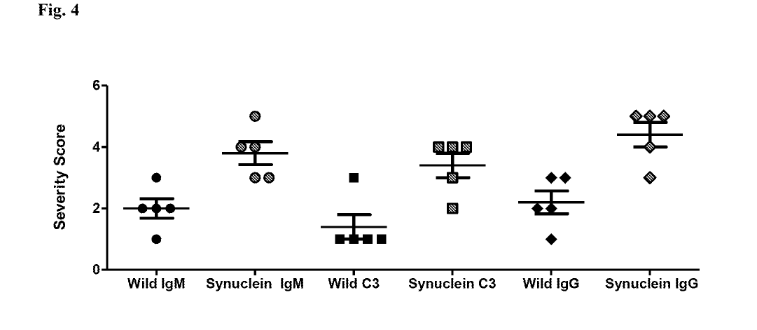

A hallmark of defective phagocytosis in vivo is the persistence of apoptotic cells which undergo necrosis leading to the production and deposition of anti-nuclear antibodies in the kidney. Antibody deposits induce complement activation and glomerular nephritis in female mice. To ascertain if the defective phagocytosis observed in vitro takes place in vivo anti-nuclear antibodies (anti-ANA) and kidney pathology were assessed in line 26 mice (see FIG. 4). Anti-nuclear antibodies were elevated in the serum of synuclein transgenic females from line 3 and line 26 animals. Kidneys from female wild type or synuclein overexpressing mice were stained for C3, IgG, and IgM and the severity of pathology was calculated by a certified pathologist. Mice overexpressing synuclein displayed increased C3, IgG, and IgM deposition in the kidney.

### Example 2

### Increases in Synuclein Levels Result in Defective Phagocytosis

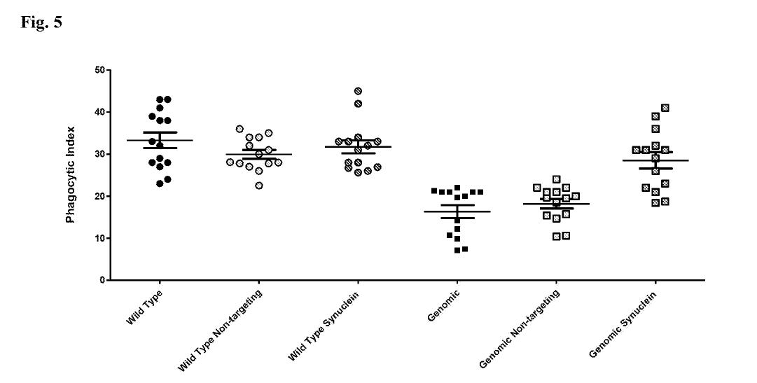

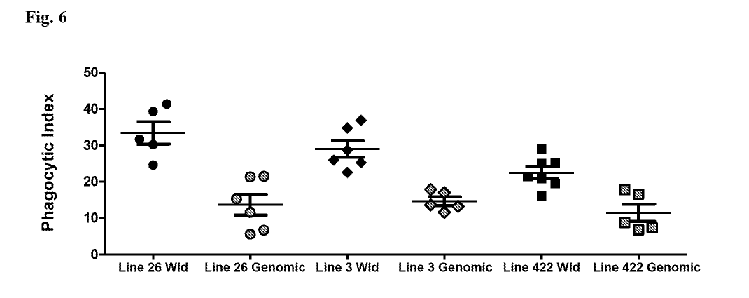

siRNA knockdown of human synuclein was employed to ascertain whether elevated synuclein protein levels were responsible for reduced phagocytosis. siRNA knockdown of human synuclein was performed on peritoneal mouse macrophages isolated from 18 month old line 26 mice and engulfment was assessed. Specific targeting of human synuclein with Accell siRNA (available from Thermo Fisher Scientific, Lafayette, Colo.) resulted in a 50-80% decrease in human synuclein mRNA, which coincided with a concomitant decrease in synuclein protein levels (see FIG. 5). siRNA knockdown of human synuclein but not treatment with non-targeting siRNA restored phagocytosis in macrophages overexpressing synuclein and had minimal activity on wild type cells. An additional concern of the genomic mice is insertion or interference by the BAC construct with a critical phagocytic gene therefore we compared the phagocytic activity of microglia isolated from 3 synuclein genomic lines overexpressing either wild type (Line 422) or the E46K synuclein mutation (line 26 and 3) (see FIG. 6). Microglia isolated from individual pups were cultured and littermate wild type or human synuclein expressing cells were examined. Over-expression of wild type or E46K human synuclein in all three lines resulted in defective microglia phagocytosis. These data indicate that the phagocytic deficits were not due to aberrant expression of genes necessary for phagocytosis and is likely due to increased levels of synuclein.

H4 cells are a human neuroglioma cell line and have two critical characteristics for our studies: ease of transfection (transfection efficiency of around 60-75%, data not shown) function to ingest latex beads. H4 cells were transfected with alpha- or beta-synuclein allowed to recover for 48 hr, then fed 6 μm beads for 90 minutes. Cells were then fixed and phagocytosed beads were counted, or the cells were lysed for western blot analysis. Alpha- but not beta-synuclein overexpression reduced phagocytosis by 50%, which mimicked data from the synuclein genomic microglia.

### Example 3

### Synuclein Point Mutants and Truncations Linked to Human Disease Alter Phagocytosis Phenotype

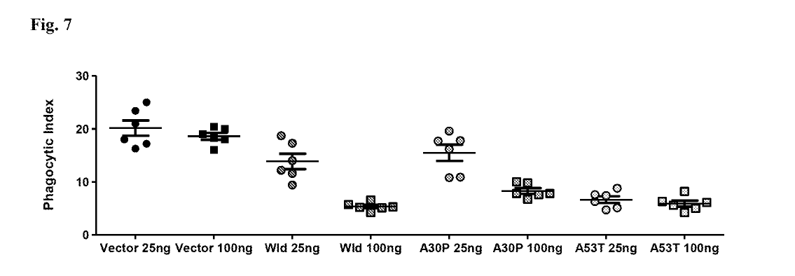

Synuclein mutations found in familial Parkinson's have been associated with more severe substantia niagra pathology and cellular defects. Therefore the relative effects of various synuclein mutants were assessed for activity in this model. A53T, E46K and A30P synuclein were all found to block phagocytosis to the same degree as wild type synuclein. A subset of these constructs, wild type, A53T, and E46K mutated synuclein were assessed for their dose dependent effect on phagocytosis. H4 cells were transfected with 30 or 90 ng/ml of the various constructs for 48 hr. Although all of the various synuclein forms were expressed to similar levels, the familial mutant A53T appeared to blocked phagocytosis more robustly than the wild type or E46K mutant at the lower dose (see FIG. 7).

In addition to assessing activity of synuclein familial mutants we also investigated the activity of truncated mutants (SN1-133 and SN1-119) which have been found in Lewy Bodies (U.S. Pat. No. 7,358,331). Transfection with the 133 truncated forms, but not the 119 truncated form blocked phagocytosis pointing to perhaps a role for the C-termini in mediating a role for synuclein in vesicle trafficking linked to phagocytosis.

### Example 4

### Altered Cytokine Release Profile with Increased Synuclein Expression

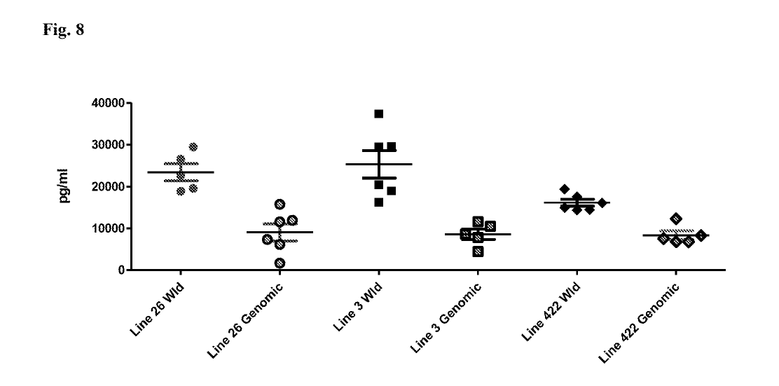

Activated microglia and increased inflammatory mediators are found in the brains of subjects with Parkinson's and are associated with synuclein overexpression in vivo. To investigate the consequence of elevated synuclein on the inflammatory response microglia from mice overexpressing human synuclein were isolate and exposed to LPS for 18 hr at which point supernatants were collected for quantification of cytokine production. Based on the increased pro-inflammatory cytokines observed in the CSF and brains of subjects with Parkinson's it was anticipated that microglia overexpressing human synuclein would have an enhanced inflammatory cytokine response. However, microglia overexpressing human synuclein secreted significantly less TNF-alpha and IL-1beta in response to LPS when compared to their littermate controls (see FIG. 8). This defect was observed in cells isolated from mice overexpressing wild type or the E46K mutation indicating that again elevated levels of synuclein rather than alterations in its phosphorylation state contribute to the defect.

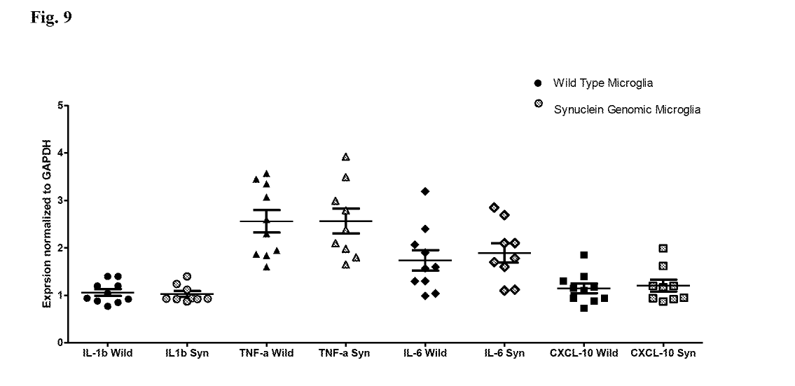

The above procedure measures only cytokines released from the cells into the media are collected. The mechanics and signaling pathway which regulate phagocytosis are also known to control exocytosis of cytokine containing vesicles. Therefore, the decreased cytokine response may reflect a defect in cytokine secretion rather than production. To investigate the possibility that the altered cytokine response was due to defective vesicle release and consequently form a linkage between the phagocytic defect and the reduced cytokine response, LPS induced signaling in wild type and synuclein genomic microglia was assessed. Induction of LPS responsive cytokines was quantified at the mRNA level by RT-PCR to investigate whether wild type and synuclein overexpressing microglia respond equivalently to LPS. For this experiment we utilized our animals which over-express human synuclein on the murine synuclein null background to limit any confounding effects of endogenous murine synuclein. mRNA from microglia stimulated with LPS for 8 hr was harvested and 12 cytokines were assessed by multiplex analysis. LPS stimulated equivalent cytokine production at the mRNA levels in synuclein null and microglia overexpressing human synuclein (see FIG. 9).

These data suggest reduced cytokine production from microglia overexpressing human synuclein is due to decreased release of cytokine containing vesicles rather than alterations in their inflammatory response. To further test the hypothesis that wild type and synuclein genomic microglia synthesize equivalent levels of cytokine protein but differ in their ability to release cytokine containing vesicles, secretion of cytokines was blocked and intracellular cytokine levels assessed. Microglia from synuclein null or human synuclein genomic/murine synuclein null pups were stimulated with LPS in the presence of absence of GOLGIPLUG (available from BD Biosciences), a reagent containing Brefeldin A, which prevents trafficking of vesicles from the endoplasmic reticulum to the Golgi thus preventing the release of cytokines into the tissue culture media. Tissue culture supernatants were collected to assess the effectiveness of Golgi Plug on cytokine release, and cells were lysed to quantify intracellular cytokine levels. As observed previously microglia overexpressing human synuclein exhibited a blunted LPS cytokine response compared to the wild type control and treatment with GOLGIPLUG blocked TNF-alpha release from both populations. When cytokine levels from the cellular lysates were evaluated microglia expressing human synuclein were found to have slightly higher intracellular TNF-alpha levels compared with wild type controls, and treatment with GOLGIPLUG significantly increased these levels. In addition following GOLGIPLUG treatment wild type and synuclein genomic samples had equivalent intracellular TNF-alpha levels, suggesting that the apparent blunted response of the genomic microglia to LPS is likely due to defective release of cytokine containing vesicle rather than defects in overall cytokine production. Although the cytokine levels are lower from the synuclein genomic microglia they are still robust enough to be pro-inflammatory and potentially detrimental to surrounding cells.

### Example 5

### Alteration in Phagocytosis is Due to Defective Vesicle Function

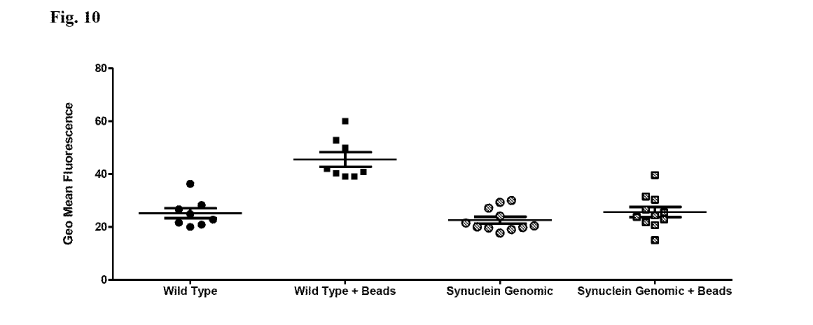

Phagocytosis of large particles requires the mobilization and addition of a significant quantity of membrane to the plasma membrane. Membrane addition comes in part from the fusion of recycling endosomes and possibly the endoplasmic reticulum with the plasma membrane. During the phagocytic process the plasma membrane of macrophages actually expands as membrane is added. This process can be traced by following membrane expansion with a plasma membrane labeling dye (FM-143), the more membrane the more dye that binds and the bigger the fluorescence signal. To assess the effect of overexpressed synuclein on vesicle fusion and membrane expansion H4s were transfected with a GFP vector or human synuclein, cells were fed beads for 90 minutes at which point cells were placed on ice labeled with FM-143 and fluorescence measured by flow cytometry. Mock vector transfect H4s displayed increased FM-143 fluorescence following bead addition, indicating vesicle fusion with and expansion of the plasma membrane. Cells overexpressing synuclein did not exhibit increased FM-143 fluorescence indicating a defect in this process (see FIG. 10). Defective FM-143 fluorescence was quantified over 4 experiments. Defective vesicle fusion during phagocytosis was also quantified in microglia from synuclein genomic mice. Our data support the notion that synuclein overexpression hinders release of cytokine containing vesicles and reduced vesicle dependent expansion of the plasma membrane during phagocytosis therefore strengthening the notion that synuclein acts as a negative regulator of vesicle function. Cells are composed of various vesicle pools, one of the most common being endosomes (early, late, and recycling) which can be identified by specific surface markers. To start tracking vesicle trafficking in synuclein overexpressing cells we took used Rab5a, a marker of early endosomes, tagged with GFP. H4's were transfected with Rab5a-GFP in conjunction with nothing or synuclein, transfected cells were fed beads for 90 minutes after which cells were fixed and stained for synuclein. Rab5a endocytic vesicles localized to sights of bead ingestion and a process blocked by over expression of synuclein. Alteration in other endosomal vesicles was assessed in synuclein overexpressing H4's. Following bead addition, H4's were fixed and stained for synuclein and SNAP23. SNAP 23 resides on vesicle pools, is involved in receptor recycling in neurons, and is a key member of SNARE complexes. Similar to Rab5a containing vesicles, endosomes with SNAP23 translocated to sights of bead addition and translocation was blocked in synuclein overexpressing cells.

H4 cells overexpressing synuclein or mock vector transfected H4s were fed beads for 90 minutes and FM-143 labeling was done on ice followed by flow cytometry analysis. (B) Geometric mean fluorescence from 4 independent experiments of H4 cells overexpressing synuclein fed beads was compiled. (C) Microglia isolated from wild type of synuclein overexpressing pups were assessed by flow cytometry for FM-143 staining prior to and after bead addition. (D) Geometric mean fluorescence of FM-143 from 3 independent was compiled. (E) H4 cells were co-transfected with GFP-tagged Rab5a, nothing, or synuclein. After 48 hr cells were fed 4 μM beads for 90 minutes. Cells were fixed and stained for synuclein. (E) H4 cells transiently transfected with mock vector or synuclein were fed 4 μM beads for 90 minute, cells were fixed and stained for SNAP23.

### Example 6

### Synuclein Inhibition of Vesicle Mobilization and Fusion is Associated with Altered SNARE Complex Formation

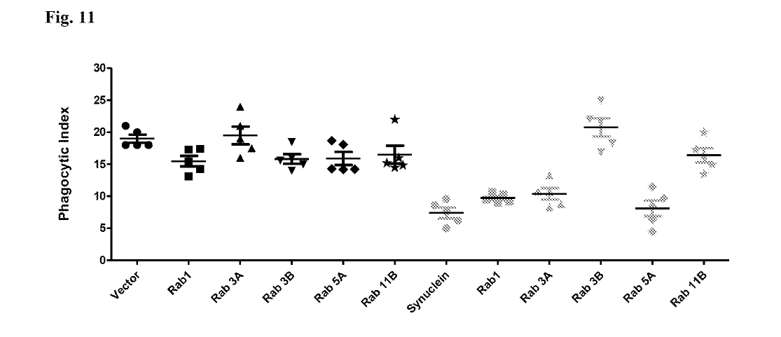

In quantifying phagocytosis in cells overexpressing synuclein we noticed that synuclein translocates to the phagocytic cup and localized with regions of active actin rearrangement. Under resting conditions synuclein resides in the cytoplasm and following 45 and 90 minutes of bead addition synuclein translocates to sites of actin polymerization and bead contact. SNARE complexes are composed of three components, a SNAP, a syntaxin, and a VAMP protein. These three proteins come together and prime vesicles for fusion with the plasma membrane. On fusion vesicles release their contents and SNARE complexes are disassembled by two adaptor proteins, NSF and α-SNAP. Some members from these dissociated complexes recycle back to the cytoplasm via recycling endosomes and can be used to prime subsequent fusion events. All three protein members of the SNARE complex are alpha helices and on assembly form an energy favorable, very unique SDS stable complex. Although these complexes resist SDS dissociation they will dissociate following boiling. Therefore one SNARE complexes can be flowed as SDS stable complexes in boiled and unboiled lysates. We hypothesized that defective vesicle mobilization may be associated with or due to altered SNARE complex formation of SNARE complex. H4 cells transfected with synuclein were fed beads for 15, 45, or 90 minutes, samples were lysed and run on western formation. Addition of beads induced rapid decrease in SNAP23 containing SNARE complexes in vector transfected cells, however no such changes were observed in synuclein overexpressing cells. Alterations in SNARE complexes were also observed in microglia isolated from synuclein genomic mice (data not shown). To elucidate how synuclein alters vesicle trafficking and SNARE complex formation/function we assessed interaction of synuclein with components of the SNARE family. Surprisingly SNAP23 and synuclein, but not SNAP 25 co-immunoprecipitated and this interaction increases following bead addition. Rab proteins are markers of endosomal vesicles and can modulate vesicle trafficking and have been shown to rescue synuclein induced toxicity in cellular modules and protect substantia niagra dopamine neurons from MTPT induced toxicity. To test the possibility that molecules which facilitate vesicle movement could overcome the ability of elevated levels of synuclein to block phagocytosis, a stable H4 cell line overexpressing synuclein was transfected with various Rab proteins. We verified that the Rab proteins were overexpressed and phagocytosis was assessed in wild type and synuclein over expressing cells. Overexpression of Rab3b and Rab11b rescued synuclein modulation of phagocytosis while overexpression of other Rab proteins had no affect (FIG. 11).

Overexpression of the delta 119 version of synuclein, which does not block phagocytosis, was assessed for its impact on vesicle fission. Overexpression of synuclein, but not the delta 119 form of synuclein blocked FM-143 addition, correlating with phagocytic activity.

### Example 7

### IL-4 Treatment Reduces Synuclein Levels and Restores Phagocytosis in Genomic Microglia

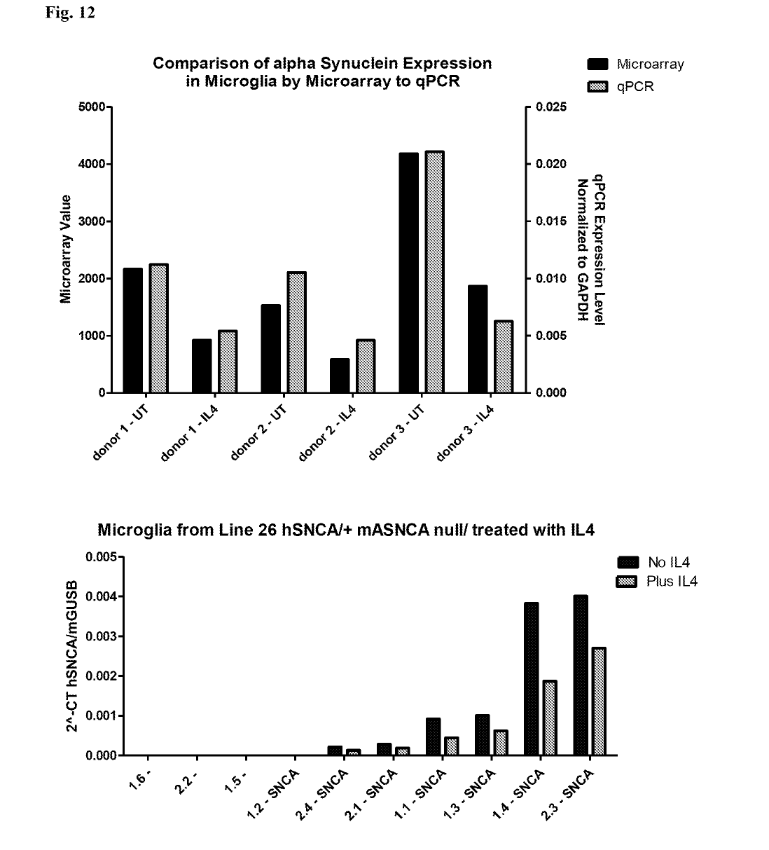

Human microglia were treated with IL-4 and synuclein transcript levels were measured by microarray and qPCR. Following IL-4 treatment synuclein levels were decreased by 50% in the three donors tested (FIG. 12). IL-4 treatment also reduced synuclein mRNA levels in line 26/synuclein null genomic microglia. As IL-4 treatment decreased synuclein transcript levels by 50% we investigated whether synuclein protein was affected by IL-4 treatments synuclein protein levels were tracked. Microglia from synuclein genomic mice were treated with IL-4 for 24 hours, and while synuclein mRNA levels were decreased, protein levels were unaffected. Synuclein is turned over slowly, therefore 24 hours maybe too soon to see affects. To assess this microglia were stimulated with IL-4 for 48 hr at which time a 50% reduction in synuclein protein levels was observed. The effect of IL-4 on synuclein mRNA and protein levels appears to be unique as other anti-inflammatory stimuli did not alter synuclein mRNA or protein. This may point to a direct effect of IL-4 signaling on the synuclein promoter.

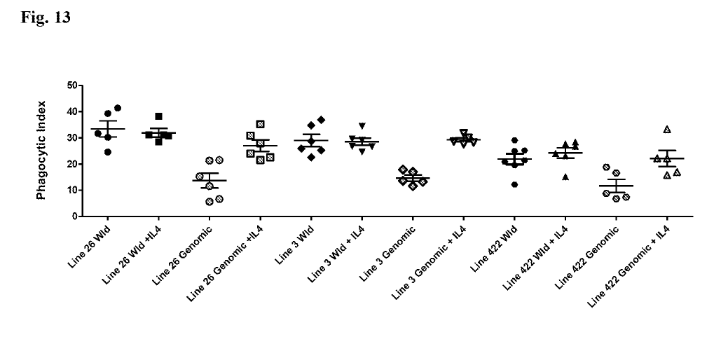

Although 24 hr of IL-4 did not decrease synuclein protein, it did rescue the phagocytic defect in microglia isolated from all synuclein genomic mice (FIG. 13). Although IL-4 decreased synuclein mRNA and protein it does not affect levels till 48 hr, whereas its ability to rescue phagocytosis occurs within 24 hr, pointing towards an additional mechanism. To determine if IL-4 worked in part by altering SNARE or vesicle movement microglia from synuclein genomic mice were treated with IL-4 for 24 hr and lysates were probed for components of the SNARE complex. IL-4 treatment increased monomer SNAP23 Syntaxin4, and SNAP23 snare complex in wild type and synuclein OE cells. After addition of beads we saw induction of SNARE complexes and these were enhanced following IL-4 treatment.

Collectively these data implicate synuclein as a negative regulator of vesicle trafficking/and or vesicle fusion. Synuclein may impair vesicle trafficking and thus alters snare function or synuclein may alter SNARE function and consequently restricts vesicle movement. The Rab data point toward synuclein altering vesicle mobilization, a process that can be overcome by addition of Rab proteins which act ad vesicle chaperones. Rab3b and Rab11b which rescued the phagocytic defect, are associated with and involved in recycling endosome pools. Rab proteins have also been shown to be critical for receptor recycling and maintenance of the recycling endosome pool. In addition Rab3 has been shown to can control the formation and stability of the snare complex possibly through modulation of NSF activity. The interaction of synuclein with SNAP23 may implicate alterations in SNARE complex activity. Dopaminergic cells are unique from other neuronal cells due in part to their use of the recycling endosome pool to prevent vesicle depletion following long term stimulation. In addition alteration of SNARE proteins and vesicle trafficking machinery is altered in Parkinson's and in vivo model of synuclein toxicity. In addition Rab over-expression is able to protect cells from synuclein induced toxicity in various in vitro models.

### Example 8

### Biomarker Assay

This example describes a flow-cytometry biomarker assay to evaluate the phagocytic index of monocytes isolated from peripheral blood (e.g., from a Parkinson's patient) and correlate the alterations in phagocytosis with concurrent increase in intracellular synuclein staining. Reduced phagocytosis in monocytes from PD patients or animal models correlates with increased synuclein levels. In this assay, peripheral macrophages are isolated from susceptible patients. Samples are assessed for phagocytosis and for synuclein levels. Defective phagocytosis acts as a biomarker to identify sporadic Parkinson's and segregate symptomatic vs. asymptomatic carriers.

The assay can be designed to distinguish internalized beads from those which are solely bound to the surface of the cells. In this assay, strepavidin labeled or biotin labeled beads are fed for 90 minutes to peripheral macrophage or monocytes isolated from patient blood at 37° C. Fluorescently labeled biotin or strepavidin is added to cells on ice to identify beads which are not phagocytosed. A separate population of isolated peripheral macrophages is identified from surface makers and intracellular synuclein staining is performed as described below. A combination of decreased phagocytosis determined from internalized beads and increased synuclein levels indicated by intracellular staining provides an indication of that symptomatic Parkinson's disease is present or imminent.

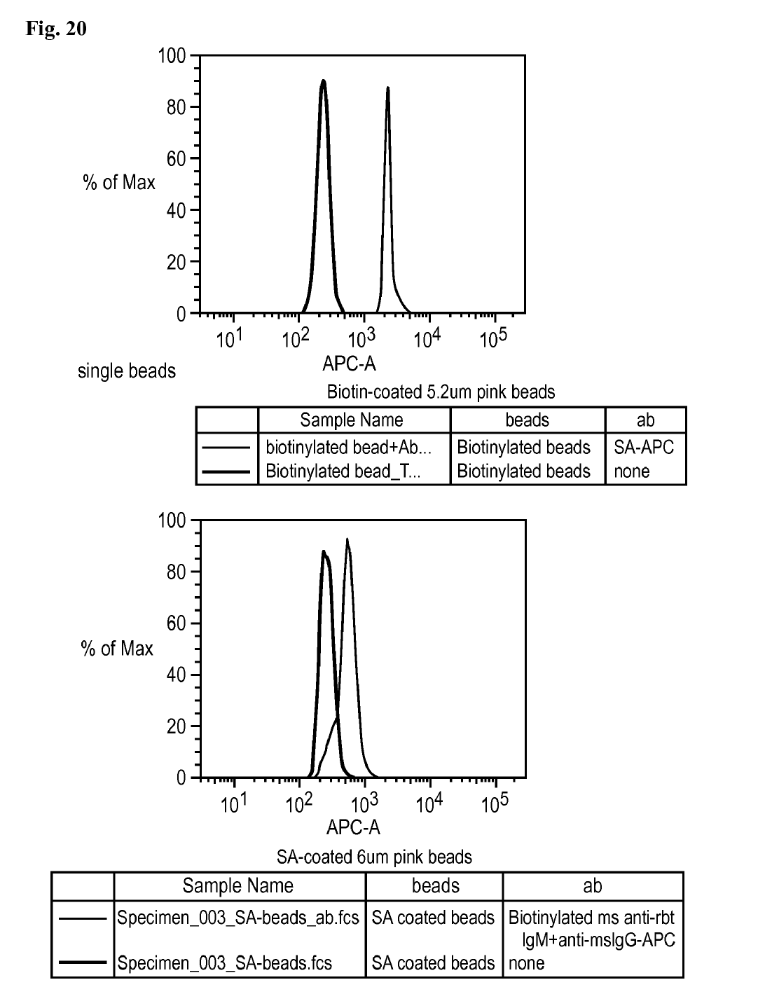

Validations of secondary detection of fluorescently labeled beads: 6 μm strepavidin-Fluorescbrite® YG microsphere (Polyscience, Cat#24157, lot588479) or 5.2 μm (pink), biotin-coated beads (Spherotech, Cat# TFP-5058-5, Lot R01; TFP-3067-5, Lot WI; TFP-2058-5, Lot R01) were tested. The beads were vortexed and briefly sonicated to prevent aggregation before aliquoting 10 μl/well of each type of bead into a 96-well V-bottom plate. The beads were washed with 100 μl/well of assay buffer (H/S±±/0.3% BSA) twice before incubating the strepavidin-coated beads with 10 μg/ml biotinylated mouse anti-rabbit-IgM (BD Biogen) or strepavidin-APC at 1:100 (R&D Systems, F0050) or assay buffer at room temperature in dark for 30 min. The beads were spun down and washed with 100 μl/well assay buffer twice. The strepavadin-coated beads were incubated with 100 μl/well anti-mouse IgG-APC at 10 μg/ml on ice, in dark for 30 min. The beads were washed with 100/well assay buffer once, then resuspended in 100 μl/well assay buffer before reading the samples. Biotin tagged fluorescent antibodies bound to and identified the strepavadin coated beads (FIG. 20, lower) and strepavadin tagged fluorescent antibodies detected biotin coated beads (FIG. 20 upper) verifying this approach as a means to detect intra vs. extracellular beads.

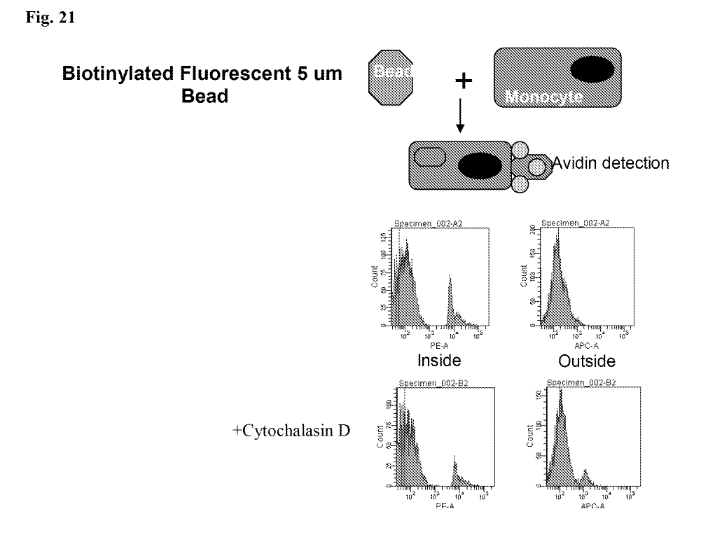

Phagocytosis assay: 6 μm strepavidin beads or 5.2 μm biotin beads were added to microglia for 90 minutes. Cells were moved to ice and stained with either a biotin or strepavidin labeled antibodies to identify un-ingested beads. To confirm the ability to identify intra-verses extracellular bound beads by flow cytometry microglia were treated with Cytochalasin D for 30 minutes prior to the addition of beads. Normal microglia were capable of engulfing 5 and 6 micron beads and Cytochalasin D treatment blocked this phagocytosis which was confirmed by biotin-APC binding to the beads (FIG. 21)

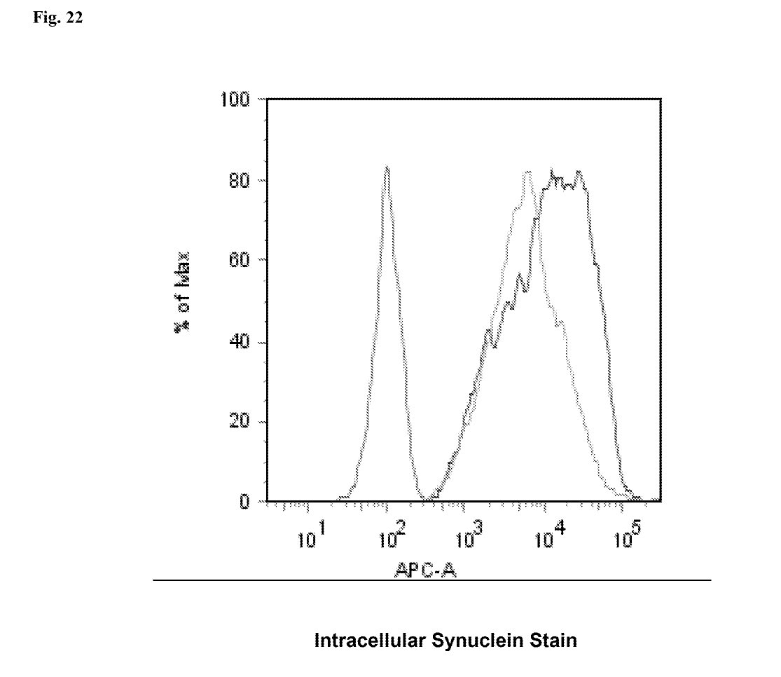

Intracellular synuclein staining: H4 cells untreated or treated with tetracycline for 24 hours to induce synuclein expression. After 24 hours cell were fixed with 4% PFA, permeabilized with 1% saponin on ice for 30 minutes. Intracellular synuclein was detected by incubating permeabilized cells with a monoclonal antibody 5C12 followed by cy3 labeled anti-mouse secondary. A two fold increase in synuclein expression was observed between synuclein overexpressing and wild type cells, similar to what we observe by western blot (FIG. 22).

### Example 9

### Overexpression of the G2019S Form of LRRK2 Enhanced Phagocytosis

Although neurons express LRRK2, this expression appears to be low relative to that of B-cells and macrophages. We found that primary microglia express LRRK2 and that LRRK2 phosphorylation can be enhanced following stimuli which engage the actin cytoskeleton (MCSF or bead addition). For example, murine microglia were stimulated with MCSF for various times or 10 uM beads for 30 minutes. LRRK2 phosphorylation was probed with PT1967 P-LRRK2 antibody.

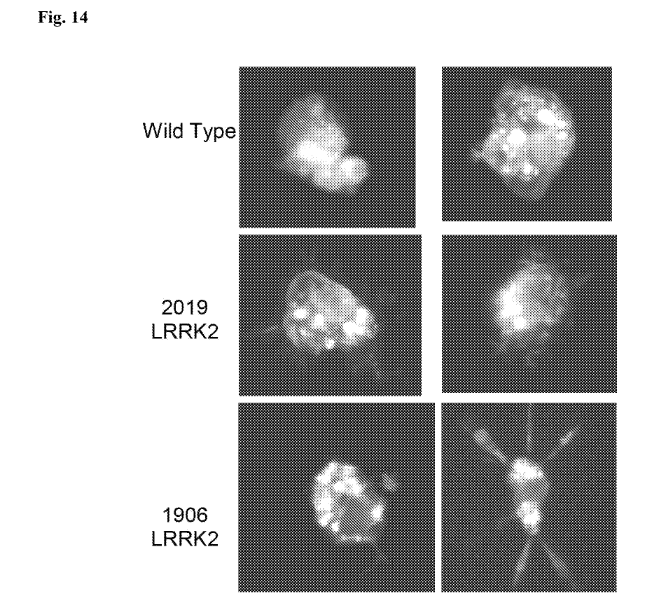

Because LRRK2 phosphorylation was apparently induced following cytoskeletal rearrangement we investigated the effect of LRRK2 kinase activity on actin cytoskeleton changes in cells nucleoporated with LRRK2 G2019S. Nucleoporation of microglia with the kinase active G2019S form of LRKK2 induces spontaneous actin rearrangement as measured by increased staining with the F-actin phalloidin dye and induction of micro spikes around the cell (FIG. 14). The increase in actin containing ruffles upon G2019S addition appeared to be due to increased kinase activity because nucleoporating wild type, 1906, or the dual 1906/2019 mutant did not induce actin cytoskeleton changes.

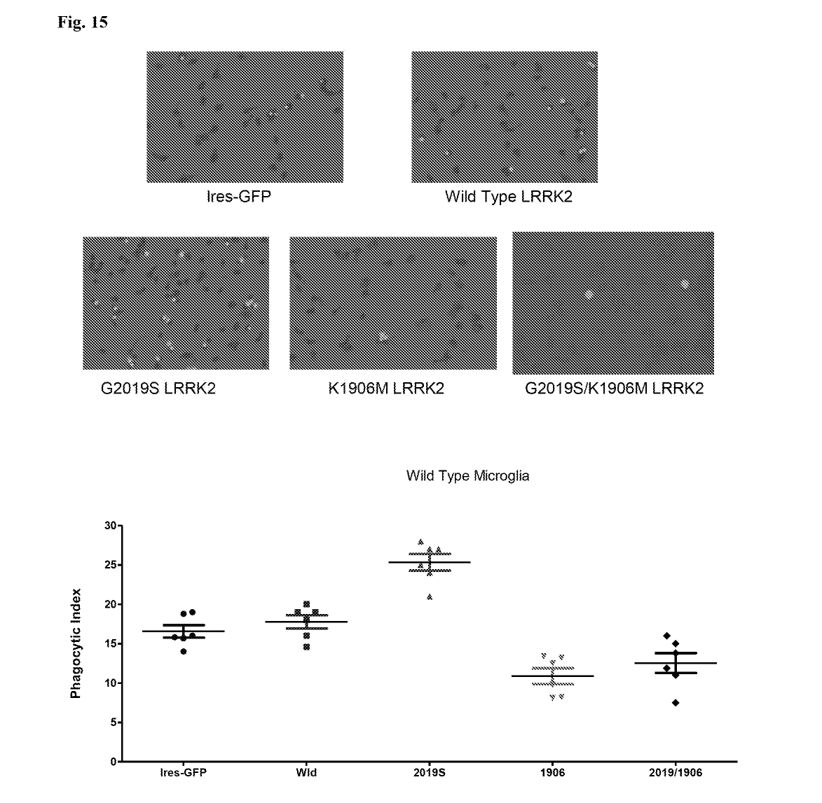

Because phagocytosis induces and requires rearrangement of the actin cytoskeleton, we hypothesized that enhanced actin cytoskeleton changes observed upon G2019S overexpression would alter microglia phagocytic activity. To assess this we fed microglia nucleoporated with various LRRK2 constructs 10 micron beads for 90 minutes and found that overexpression of the G2019S form of LRRK2 enhanced phagocytosis while the wild type form had no affect (FIG. 15). Overexpression of the kinase inactive LRRK2 1906 and 1906/2019 seemed to reduce phagocytosis possibly acting as a dominant negative partner with in a LRRK2 homodimer.

Our observations regarding increased actin polymerization and increased micro spikes on the cells in addition to elevated phagocytosis following G2019S nucleoporation implied that Rac activity may be elevated in a LRRK2 kinase dependent manner. To investigate this hypothesis we assessed the basal activation of Rac in HEK cells stably transfected with wild type, G2019S, or 1906 LRRK2. Cells stably transfected with the G2019S form of LRRK2 had elevated spontaneous rac activity while cells expressing either wild type or 1906 form of LRRK2 did not. These data taken together indicate that the kinase activity of LRRK2 is associated with the induction of actin cytoskeleton rearrangement resulting in increased phagocytosis likely due to elevated Rac activity.

### Example 10

### A Cellular Assay for Testing Potency of Compounds on LRRK2 Kinase Activity

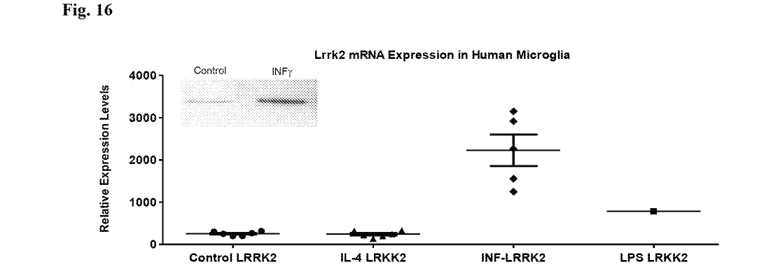

We have tested the effect of LRRK2 kinase inhibitors on phagocytosis in cells transformed with LRRK2 G2019S. We have a number of compounds known to inhibit LRRK2 kinase activity. Some of these compounds have enantiomers that are known inhibitors of either cFMS or PLK2 inhibitors but have minimal effect on LRRK2. Pairs of enantiomers one of which is known inhibitor of LRRK2 and one of which inhibits cFMS or PLK2 without substantially effect on LRRK2 can be used to measure LRRK2-specific effects on phagocytosis. We found that INF-γ treatment of microglia induced LRRK2 mRNA transcript levels and protein levels by about 2 fold (FIG. 16) and the increase in LRRK2 levels coincided with an apparent increase in LRRK2 kinase activity as measured by increased LRRK2 phosphorylation as well as increased kinase activity in a LRRK-tide phosphorylation assay.

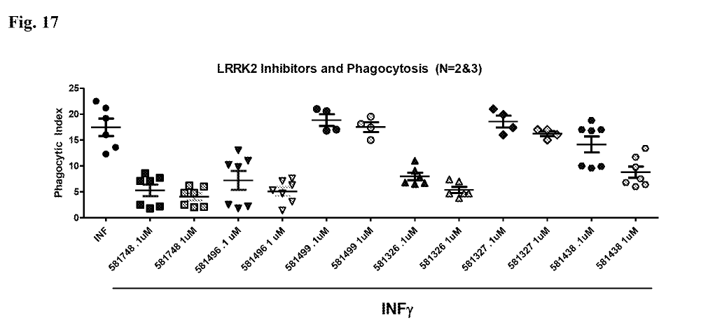

Treatment of microglia for 24 hr with INFγ increased phagocytosis whereas treatment of cells with INFγ for 24 followed by a 20 minute treatment with the various LRRK2 inhibitors reduced the phagocytosis of 10 micron bead (FIG. 17). Reduced phagocytosis was not observed in cells treated with the LRRK2 inactive enantiomer inhibitors. These results indicate that LRRK2 inhibitors reduce phagocytosis in cells having elevated levels of phagocyotosis. At first site, these results appear anomalous because if reduced phagocytosis is associated with Parkinson's disease and LRRK2 inhibitors are useful for treating Parkinson's disease, one might expect LRRK2 inhibitors to increase phagocytosis. The apparent anomaly is reconciled in the following example, which shows that LRRK2 has opposing effects on phagocytic activity depending whether or not cells overexpress synuclein, as is the case in Parkinson's disease.

### Example 11

### The Effects of LRRK2 Kinase Activity and Synuclein Levels on Phagocytic Activity

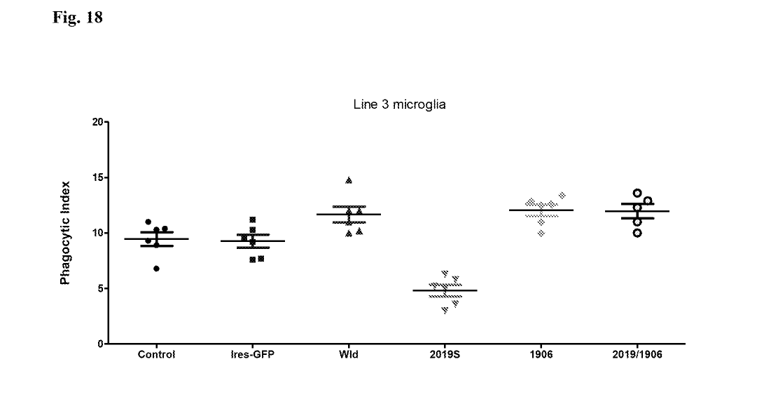

Because both LRRK2 kinase activity and elevated synuclein levels impact phagocytic activity we tested the effect of combining synuclein overexpression and increased LRRK2 kinase activity. Microglia isolated from animals homozygous for a human alpha synuclein transgene were nucleoporated with various LRRK2 constructs, and after 4 days in culture microglia were fed 10 μM beads for 90 minutes and a phagocytic index calculated. Surprisingly unlike, in wild type microglia in which the G2019S form of LRRK enhanced phagocytosis, in synuclein overexpressing cells it significantly decreased phagocytosis (FIG. 18). The effect of the G2019S construct on phagocytosis appears to be due to increased kinase activity because the 1906 and 2019/1906 constructs had no effect.

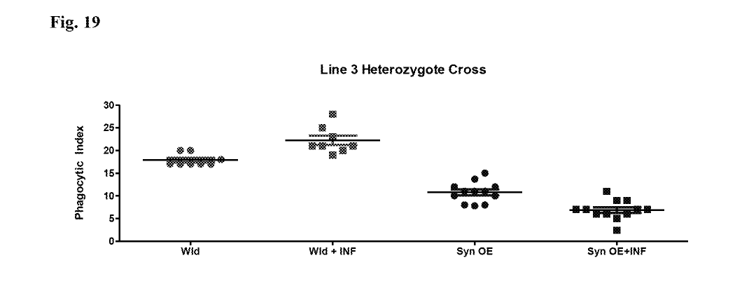

To test a more physiologic system in which altered activation of LRRK2 and elevated synuclein may occur, we tested the effect of INFγ treatment on phagocytosis in wild type and synuclein-overexpressing microglia. As described above INFγ treatment naturally raises LRRK2 levels as well as increased LRRK2 phosphorylation and activity. Using our line 3 heterozygote X heterozygote crosses (generating homozygous alpha synuclein mice) in which, by culturing each pup separately wild type and synuclein overexpressing littermate cultures are generated. Microglia isolated from the various pups were exposed to INFγ overnight and which point 10 uM beads were added for 90 minutes and phagocytosis was assessed while we were still blinded to the genotype of each culture. Surprisingly we observed that INFγ treatment modulated phagocytic activity similarly to the G2019S LRRK2 construct. INFγ treatment of wild type microglia enhanced phagocytosis whereas this same treatment on synuclein overexpressing cells resulted in an even further reduction in phagocytosis (FIG. 19). These data supply evidence that environmental factors, in particular inflammatory stimuli can modulate the function of familial Parkinson's genes, and under the correct condition may exacerbate defects which already exist due either to genetics, age, or additional environmental factors. The ability of the environment to impact these pathways may help explain the incomplete penetrance and onset of Parkinson's symptoms observed with various familial forms of PD.

All patent filings, other publications, accession numbers and the like cited above are incorporated by reference in their entirety for all purposes to the same extent as if each individual item were specifically and individually indicated to be so incorporated by reference. If different variants of a sequence are associated with an accession number at different times, the version associated with the accession number at the effective filing date of this application is meant. The effective filing date refers to the earliest of the actual filing date or filing date of any priority application disclosing the relevant accession number. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

1. A method of monitoring synucleinopathic disease or providing an indication of presence, susceptibility to or severity of synucleinopathic disease in a subject, comprising: determining phagocytic activity in a blood sample or other phagocytic cells from a subject; wherein the phagocytic activity is used in monitoring or to provide an indication of presence, susceptibility or severity of synucleinopathic disease in the subject.

2. The method of claim 1,
(a) wherein phagocytic activity is determined in a blood sample from the subject and the method comprises: comparing the phagocytic activity of the blood sample from the subject to one or more control levels of phagocytic activity, wherein reduced phagocytic activity in the subject relative to phagocytic activity of a blood sample from an undiseased individual is an indicator of presence, susceptibility or extent of synucleinopathic disease;
(b) wherein the method further comprises contacting the peripheral macrophages of the subject with IL-4 or IL-13 or agonist thereof, and assessing whether the phagocytic activity increases in response to the IL-4 or IL-13 or agonist thereof;
(c) wherein the method comprises determining phagocytic activity of blood samples or other phagocytic cells from the subject at a plurality of times, and associating a changes in phagocytic activity, if any, with changed susceptibility to or severity of the disease.

3. The method of claim 1 or 2,
(a) wherein the individual has at least one sign or symptom of synucleinopathic disease and the reduced phagocytic activity in combination with the symptom is used to diagnose the subject with synucleinopathic disease;
(b) wherein the subject lacks symptoms of synucleinopathic disease, and the reduced phagocytic activity is used in assessing susceptibility to synucleinopathic disease;
(c) wherein the subject has been diagnosed with synucleinopathic disease, and the reduced phagocytic activity is used to assess severity of synucleinopathic disease;
(d) wherein the method further comprises contacting the peripheral macrophages of the subject with IL-4 or IL-13 or agonist thereof, and assessing whether the phagocytic activity increases in response to the IL-4 or IL-13 or agonist thereof.

4-6. (canceled)

7. The method of claim 1 for monitoring synucleinopathic disease in subject, comprising determining phagocytic activity of blood samples or other phagocytic cells from the subject at a plurality of times, and associating a changes in phagocytic activity, if any, with changed susceptibility to or severity of the disease.

8. The method of claim 7, wherein the subject has been diagnosed with synucleinopathic disease and increased phagocytic activity over time is associated with reduced severity of disease.

9. The method of claim 7 or 8, wherein the subject is being treated with a drug and the plurality of times includes a time before and after initiating administration of the drug, and increased phagocytic activity indicates a positive response to the drug in the subject.

10. The method of claim 7, wherein the subject has not been diagnosed with synucleinopathic disease on commencing monitoring, and decreased phagocytic activity over time is associated with increased susceptibility to disease.

11. The method of claim 7, 8 or 10, wherein the phagocytic activity is determined in the blood sample and alpha synuclein expression in cells from the blood sample is also determined; wherein the phagocytic activity and/or the level of alpha synuclein provide an indication of susceptibility or severity of the synucleinopathic disease.

12. The method of claim 1, wherein alpha synuclein expression in cells of a blood sample from the subject or other phagocytic cells from the subject is also determined;
wherein the phagocytic activity and/or the level of alpha synuclein provides an indication of presence, susceptibility to or severity of synucleinopathic disease.

13. The method of claim 12, wherein the alpha synuclein expression and phagocytic activity are determined in a blood sample.

14. The method of claim 13, wherein
(i) the phagocytic activity and the alpha synuclein expression are determined on the same blood sample; or
(ii) the method further comprises determining the level of alpha synuclein in cells of the blood sample.

15. (canceled)

16. The method of claim 12 or 13, wherein
(i) the phagocytic activity and the synuclein expression are determined on the same or overlapping population of cells in the blood sample;
(ii) wherein the cells include peripheral macrophages; or
(iii) wherein the cells include polymorphonuclear cells.

17-18. (canceled)

19. The method of any one of claim 12 or 13, wherein the phagocytic activity is determined from a first fluorescent signal and the expression of alpha synuclein from a second fluorescent signal.

20. The method of any one of claim 19, wherein the first and second fluorescent signals are detected simultaneously.

21. The method of any one of claim 19 or 20, wherein the first and second fluorescent signals are detected by flow cytometry.

22. The method of any one of claims 19 and 20, wherein the phagocytic activity is determined from uptake of fluorescently labeled cells or beads and the alpha synuclein expression is determined from uptake of a fluorescently labeled antibody that specifically binds to intracellular alpha synuclein.

23. The method of any one of claims 12-14 and 20, wherein the method further comprises
(i) comparing the phagocytic activity of the blood sample in the subject to one or more control levels of phagocytic activity of a blood sample from an undiseased individual;
(ii) comparing the alpha synuclein expression of the blood sample in the subject to one or more control levels of alpha synuclein expression of a blood sample from an undiseased individual; or
(iii) comparing the phagocytic activity and the alpha synuclein expression of the blood sample in the subject to one or more control levels of phagocytic activity and alpha synuclein expression of a blood sample from an undiseased individual.

24-25. (canceled)

26. The method of claim 23, wherein the comparing is performed in a computer programmed to perform the comparing and provide output of an indication of presence, susceptibility to or severity of synucleinopathic disease.

27. The method of claim 1, wherein
(i) the synucleinopathic disease is sporadic Parkinson's disease;
(ii) the subject is a G2019S non-carrier; or
(iii) the subject is a G2019S carrier.

28-29. (canceled)

30. The method of claim 27, wherein the subject is a G2019S non-carrier or carrier, and wherein
(i) reduced phagocytic activity and/or increased alpha synuclein expression in the subject relative to phagocytic activity and alpha synuclein expression of a blood sample from an undiseased individual is an indicator of presence, susceptibility or extent of synucleinopathic disease;
(ii) the individual has at least one sign or symptom of synucleinopathic disease and the reduced phagocytic activity and/or increased synuclein expression in combination with the symptom is used to diagnose the subject with synucleinopathic disease; or
(iii) the subject lacks symptoms of synucleinopathic disease, and the reduced phagocytic activity and/or increased alpha synuclein expression is used in assessing susceptibility to synucleinopathic disease; or
(iv) the subject has been diagnosed with synucleinopathic disease, and the reduced phagocytic activity and/or increased alpha synuclein expression is used to assess severity of synucleinopathic disease.

31-33. (canceled)

34. The method of claim 27, wherein the subject is a G2019S carrier and wherein
(i) reduced phagocytic activity provides an indication that the subject has symptomatic synucleinopathic disease; or
(ii) increased phagocytic activity provides an indication the subject does not have symptomatic synucleinopathic disease.

35-37. (canceled)

38. A method of screening a test agent for activity useful in treatment of synucleinopathic disease comprising: contacting a test agent with a phagocytic cell containing an exogenous gene expressing alpha synuclein or from a subject with synucleinopathic disease; and determining whether the test agent increases the phagocytic activity of the cell, an increase providing an indication that the agent is useful in treatment of synucleinopathic disease.

39-43. (canceled)

44. A method of differentially treating subjects with a synucleinopathic disease, comprising: determining phagocytic activity in phagocytic cells or a blood sample of the subjects; and treating subjects with below normal phagocytic activity with a regime and treating subjects with normal or above normal phagocytic activity with a different regime.

45. A method for selecting candidate human subjects for participation in a clinical trial involving a drug for treating a synucleinopathic disease, comprising: determining the phagocytic activity of phagocytic cells or a blood sample of the human subjects, and segregating the subjects for inclusion or exclusion in the trial based on the level of phagocytic activity.

46-47. (canceled)

48. A method of screening an LRRK2 binder or modulator comprising:
(a) contacting a test agent with a phagocytic cell over-expressing alpha synuclein having reduced phagocytic activity relative to a control cell without alpha synuclein overexpression; and determining whether the test agent increases the phagocytic activity of the cell, an increase providing an indication that the agent is useful in inhibiting LRRK2; or
(b) contacting a test agent with a phagocytic cell having an LRRK2 2019 mutation and/or treated with IFN-γ, wherein the phagocytic cell does not overexpress alpha synuclein and has increased phagocytic activity relative to a control cell without an LRRK2 2019 mutation and not treated with IFN-γ; and determining whether the test agent decreases the phagocytic activity of the cell, a decrease providing an indication that the agent is useful in inhibiting LRRK2.

49-55. (canceled)

56. A diagnostic kit, comprising an entity that can be phagocytosed and an antibody to alpha synuclein.

57. (canceled)

58. A method of providing an indication of presence, susceptibility to or severity of synucleinopathic disease, comprising:
determining phagocytic activity of peripheral macrophages of a subject;
comparing the phagocytic activity of peripheral macrophages in the subject to one or more control levels of phagocytic activity, wherein reduced phagocytic activity in the subject relative to phagocytic activity of peripheral macrophages from an undiseased individual is an indicator of presence, susceptibility or extent of synucleinopathic disease.

59-62. (canceled)

63. A method of providing an indication of presence, susceptibility or severity of synucleinopathic disease, comprising determining phagocytic activity of (i) peripheral macrophages of a subject; or (ii) a blood sample from a subject in the presence and absence of IL-4 or IL-13 or an agonist thereto, wherein increased activity in the presence of IL-4 or IL-13 or agonist is an indication of the presence, susceptibility or severity of synucleinopathic disease.

64. (canceled)

65. A method of monitoring synucleinopathic disease in subject, comprising determining phagocytic activity of peripheral macrophages of the subject at a plurality of times, and associating a change in phagocytic activity, if any, over time with a change in susceptibility or severity of disease.

66-69. (canceled)

70. A method of treating or effecting prophylaxis of synucleinopathic disease, comprising administering to a subject having or at known risk of synucleinopathic disease IL-4 or a nucleic acid encoding IL-4 or an agonist of IL-4.

71. (canceled)

72. A method of treating or effecting prophylaxis of synucleinopathic disease, comprising administering rab3b or rab11b or a nucleic acid encoding either of these, or an agonist of either of these.

73. (canceled)

74. A method of providing an indication of presence, susceptibility to or severity of synucleinopathic disease, comprising determining phagocytic activity of phagocytic cells of a subject, wherein alpha synuclein expression in the phagocytic cells is also determined; wherein the phagocytic activity and/or the level of alpha synuclein provide an indication of presence, susceptibility to or severity of synucleinopathic disease.

75-94. (canceled)
